(19) United States
(12) Patent Application Publication
(10) Pub. No.: US 2007/0059254 A1
(43) Pub. Date: Mar. 15, 2007

(54) Compositions for delivering 5-ht agonists across the oral mucosa and methods of use thereof

(75) Inventor: Nikhilesh N. Singh, Mill Valley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TRANSORAL PHARMACEUTICALS, INC., CORTE MADERA, CA (US)

(21) Appl. No.: 10/567,658

(22) PCT Filed: Aug. 18, 2004
(86) PCT No.: PCT/US04/27156

§ 371 Date: Oct. 27, 2006

Related U.S. Application Data
(60) Provisional application No. 60/560,748, filed on Aug. 21, 2003.
(60) Provisional application No. 60/598,672, filed on Aug. 3, 2004.

(51) Int. Cl. A61K 9/68; A61K 31/4184; A61K 31/41

(52) U.S. Cl. 424/48; 424/400; 514/381

(57) ABSTRACT

The present invention provides novel compositions for the delivery of a 5-hydroxytryptamine (5-HT) agonist across the oral mucosa. In particular, the buffer system in the compositions of the present invention raises the pH of saliva to a pH greater than about 9.9, thereby facilitating the substantially complete conversion of the 5-HT agonist from its ionized to its unionized form. As a result, the dose of 5-HT agonist is rapidly and efficiently absorbed by the oral mucosa. Furthermore, delivery of the 5-HT agonist across the oral mucosa advantageously bypasses hepatic first pass metabolism of the drug and avoids enzymatic degradation of the drug within the gastrointestinal tract. Methods for using the compositions of the present invention for treating migraines are also provided.

### CROSS-REFERENCES TO RELATED APPLICATIONS

This application claims priority to each of U.S. Ser. No. 10/646,659, filed Aug. 21, 2003 and U.S. Ser. No. 60/598,672 filed Aug. 3, 2004 (Atty Docket No. 022205-000310US), the disclosures of each being incorporated herein by reference.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

While there are various types of dosage forms, solid dosage forms for oral administration are perhaps among the most preferred by patients, and among the most prevalently used. These dosage forms are typically medicaments formulated as tablets, capsules, or liquids, which are swallowed. Oral administration, however, has several disadvantages, such as drug losses during hepatic first pass metabolism, during enzymatic degradation within the GI tract, and during absorption. These drug losses not only increase the variability in drug response, but also often require that the medicament be given in greater initial doses. In addition, because the drug has to pass through the gastrointestinal system in order to enter the blood stream, the time to reach a therapeutic effect may be quite long, typically around forty-five minutes or longer.

Accordingly, other routes of drug administration have been investigated, including those involving transport across the mucous membranes. Of the various mucous membranes (e.g., oral, rectal, vaginal, ocular, nasal, etc.), drug delivery via the mucous membranes in the oral cavity seems to be the most easily tolerated by patients. In addition to avoiding the problems with traditional oral administration, drug delivery via the mucous membranes of the oral cavity has certain other advantages, due to the properties of the oral mucosa itself. For example, the mucous membranes of the oral cavity are highly vascularized and well supplied with lymphatic drainage sites.

In general, the mucous membranes of the oral cavity can be divided into five main regions: the floor of the mouth (sublingual), the cheeks (buccal), the gums (gingival), the roof of the mouth (palatal), and the lining of the lips. These regions differ from each other with respect to their anatomy, drug permeability, and physiological response to drugs. For example, in terms of permeability, sublingual is more permeable than buccal, which is more permeable than palatal. This permeability is generally based on the relative thickness and degree of keratinization of these membranes, with the sublingual mucosa being relatively thin and non-keratinized, the buccal mucosa being thicker and non-keratinized, and the palatal mucosa being intermediate in thickness, but keratinized.

In addition to the differences in permeability of the various mucous membranes, the extent of drug delivery is also affected by the properties of the drug to be delivered. The ability of a molecule to pass through any mucous membrane is dependent upon its size, its lipid solubility, and the extent to which it is ionized, among other factors.

The extent to which a drug is ionized has further been investigated with respect to drug delivery across the mucous membranes. Ionization is dependant on the dissociation constant, or pKa of the molecule, and the pH of the molecule's surrounding environment. In its un-ionized form, a drug is sufficiently lipophilic to traverse a membrane via passive diffusion. In fact, according to the pH partition hypothesis, only un-ionized, non-polar drugs will penetrate a lipid membrane.

At equilibrium, the concentrations of the un-ionized form of the drug are equal on both sides of the membrane. As the concentration gradient drives passive diffusion, an increase in the percentage of the un-ionized form of a drug correspondingly increases the transmucosal absorption of the drug. Maximum absorption across the membrane is thought to occur when a drug is 100% in its un-ionized form. Similarly, absorption across the membrane decreases as the extent of ionization increases. Therefore, one may influence the extent of drug absorption across the mucous membranes of the oral cavity by altering the salivary pH.

Some of the known transmucosal dosage forms include the use of a single buffering agent in order to change the pH of the saliva and tissues surrounding the buccal mucosa. However, these single buffering agents typically react with an acid or a base to create a final pH that is dependent upon the initial pH of the saliva of the user. A buffering agent used to attain a final pH that is dependent upon the initial pH of the user results in great variability. The extent of ionization, and hence the extent of absorption across the mucous membranes cannot be predicted with any sort of accuracy. This may pose significant problems when calculating precise doses, minimizing variability in patient response, and proving consistency in drug loading to the regulatory authorities. In addition, a single buffering agent is typically not capable of sustaining a given pH over a period of time for optimal absorption. While others in the art have disclosed the use of more than one buffering agent, these aforementioned problems are not easily cured by the nonchalant addition of an extra buffering agent, which may be unsafe and cause irreversible damage to the mucous membranes of the oral cavity. As such, a buffering system capable of achieving and sustaining a final pH independent of the initial pH in order to increase transmucosal absorption has not heretofore been demonstrated.

Similarly, a buffer system that facilitates substantially complete conversion of the ionized form of a drug to the un-ionized form in the shortest period of time, which is critical for producing rapid delivery of practically an entire drug dose across the oral mucosa, has not heretofore been demonstrated. Previous dosage forms resulted in great variability in drug delivery, due to the variability in the rates in which a drug was released from its carrier. That is, the rates of drug release in previously described chewing gums or lozenges are largely dependent upon the rate of chewing or sucking of the user. The variability in these rates from user to user further exacerbates the ability to predict the final amount of drug that will enter systemic circulation. In addition, the rate of drug release from chewing gums is further dependent upon the ability of the drug to be released from the gum base. Often times, the gum base strongly adheres to the drug, making portions of the drug unavailable for absorption.

Accordingly, there is a need in the art for compositions for delivering therapeutic agents across the oral mucosa having buffer systems that facilitate absorption of the agents in a safe and stable manner. Similarly, there is a need in the art for compositions for delivering therapeutic agents across the oral mucosa having a buffer system that produces a final pH, independent of the initial pH, and sustains that final pH for a given period of time. In addition, there is a need in the art for compositions capable of rapidly facilitating substantially complete conversion of a therapeutic agent from its ionized to its un-ionized form. The present invention satisfies these and other needs.

### BRIEF SUMMARY OF THE INVENTION

The present invention provides novel compositions for the delivery of a 5-hydroxytryptamine (5-HT) agonist across the oral mucosa. In particular, the buffer system in the compositions of the present invention raises the pH of saliva to a pH greater than about 9.9, thereby facilitating the substantially complete conversion of the 5-HT agonist from its ionized to its un-ionized form. As a result, the dose of 5-HT agonist is rapidly and efficiently absorbed by the oral mucosa. Furthermore, delivery of the 5-HT agonist across the oral mucosa advantageously bypasses hepatic first pass metabolism of the drug and avoids enzymatic degradation of the drug within the gastrointestinal tract. Methods for using the compositions of the present invention for treating migraines are also provided.

As such, in one aspect, the present invention provides a composition for delivery of a 5-HT agonist across the oral mucosa, the composition comprising:
(a) a 5-HT agonist or a pharmaceutically acceptable salt thereof;
(b) a carrier; and
(c) a ternary buffer system comprising a carbonate salt, a bicarbonate salt, and a metal oxide,
   wherein the ternary buffer system raises the pH of saliva to a pH greater than about 9.9 irrespective of the starting pH of saliva.

In another aspect, the present invention provides a composition for delivery of a 5-HT agonist across the oral mucosa, the composition comprising:
(a) a 5-HT agonist or a pharmaceutically acceptable salt thereof;
(b) a carrier; and
(c) a ternary buffer system comprising a carbonate salt, a bicarbonate salt, and a citrate, phosphate, or borate salt,
   wherein the ternary buffer system raises the pH of saliva to a pH greater than about 9.9 irrespective of the starting pH of saliva.

In yet another aspect, the present invention provides a composition for delivery of a 5-HT agonist across the oral mucosa, the composition comprising:
(a) a 5-HT agonist or a pharmaceutically acceptable salt thereof;
(b) a carrier; and
(c) a buffer system comprising a carbonate salt or a bicarbonate salt and two or more buffering agents selected from the group consisting of a metal oxide, a citrate salt, a phosphate salt, and a borate salt,
   wherein the buffer system raises the pH of saliva to a pH greater than about 9.9 irrespective of the starting pH of saliva.

In still yet another aspect, the present invention provides a composition for delivery of a 5-HT agonist across the oral mucosa, the composition comprising:
(a) a 5-HT agonist or a pharmaceutically acceptable salt thereof;
(b) a carrier; and
(c) a binary buffer system comprising a carbonate salt or a bicarbonate salt and a metal oxide,
   wherein the binary buffer system raises the pH of saliva to a pH greater than about 9.9 irrespective of the starting pH of saliva.

In a further aspect, the present invention provides a composition for delivery of a 5-HT agonist across the oral mucosa, the composition comprising:
(a) a 5-HT agonist or a pharmaceutically acceptable salt thereof;
(b) a carrier; and
(c) a binary buffer system comprising a carbonate salt or a bicarbonate salt and a citrate, phosphate, or borate salt,
   wherein the binary buffer system raises the pH of saliva to a pH greater than about 9.9 irrespective of the starting pH of saliva.

In another aspect, the present invention provides a composition for delivery of a 5-HT agonist across the oral mucosa, the composition comprising:
(a) a 5-HT agonist or a pharmaceutically acceptable salt thereof;
(b) a carrier; and
(c) a binary buffer system comprising a metal oxide and a citrate, phosphate, or borate salt,
   wherein the binary buffer system raises the pH of saliva to a pH greater than about 9.9 irrespective of the starting pH of saliva.

In yet another aspect, the present invention provides a method for treating a migraine in a subject in need thereof, the method comprising:

administering to the subject a composition comprising a therapeutically effective amount of a 5-HT agonist or a pharmaceutically acceptable salt thereof, a carrier, and a ternary buffer system comprising a carbonate salt, a bicarbonate salt, and a metal oxide, wherein the ternary buffer system raises the pH of saliva to a pH greater than about 9.9 irrespective of the starting pH of saliva.

Other objects, features, and advantages of the present invention will be apparent to one of skill in the art from the following detailed description and figures.

### BRIEF DESCRIPTION OF THE DRAWINGS

FIG. 1 shows the mean plasma concentration over time for Formulation A (25 mg buccal sumatriptan succinate solution) and Formulation B (25 mg Imitrex® oral tablet).

FIG. 2 shows the pH stability of a 9 mg and a 12.5 mg sublingual sumatriptan composition of the present invention.

### DETAILED DESCRIPTION OF THE INVENTION

I. Definitions

As used herein, the following terms have the meanings ascribed to them unless specified otherwise.

The term “migraine” refers to an intense, throbbing, typically unilateral headache characterized by sharp pain and often accompanied by symptoms such as nausea, vomiting, sensitivity to light and/or sound, stuffy or runny nose, watery eyes, dizziness, mood changes, allodynia, and visual disturbances. Migraines are typically recurring headaches, and most migraine sufferers (i.e., subjects) experience at least one migraine attack a month. Migraines can happen at any time and, if left untreated, can last from about 4 hours to about 3 days. One skilled in the art will appreciate that migraine symptoms can not only vary between subjects, but can also vary between migraine attacks in a given subject. The pain from a migraine is moderate to severe in intensity and can affect one of both sides of the head as well as other areas such as the back of the neck, the face, the eyes, and the sinuses. Types of migraine suitable for treatment with the compositions of the present invention include, without limitation, migraine without aura and migraine with aura. The term “migraine without aura” refers to the most common type of migraine and includes an intense, throbbing headache that is typically accompanied by sensitivity to light and/or sound. The term “migraine with aura” refers to a type of migraine that is preceded by aura.

As used herein, the term “aura” refers to the visual disturbances that some migraine sufferers have before a migraine attack. Aura typically develops gradually immediately preceding a migraine and lasts less than about an hour. Generally, about 3 out of every 10 subjects who suffer from migraines experience aura before a migraine attack. Aura is accompanied by symptoms including, without limitation, visual changes such as tunnel vision, blind spots, blurred vision, seeing flashing lights, seeing jagged lines, seeing spots, and difficulty in focusing; sensory or motor changes such as numbness or tingling of the lips, face, or hands on one or both sides and weakness in the arms and/or legs on one or both sides; and speech or language changes such as the inability to understand words, loss of speech, and the inability to speak normally.

The terms “therapeutic agent” and “drug” are used interchangeably herein to refer to a substance having a pharmaceutical, pharmacological, psychosomatic, or therapeutic effect. Preferably, the therapeutic agent or drug is a 5-hydroxytryptamine (5-HT) agonist. Suitable 5-HT agonists for use in the present invention include, without limitation, sumatriptan, naratriptan, rizatriptan, eletriptan, almotriptan, zolmitriptan, frovatriptan, F 11356, pharmaceutically acceptable salts thereof, and combinations thereof. In a particularly preferred embodiment, the 5-HT agonist is sumatriptan, in all suitable forms. In other embodiments of the present invention, the therapeutic agent or drug is a combination of a 5-HT agonist and a 5-HT antagonist. Suitable 5-HT antagonists for use in the present invention include, without limitation, ondansetron, palonosetron, tropisetron, lerisetron, alosetron, granisetron, dolasetron, bemesetron, ramosetron, azaseteron, itasetron, zacopride, cilasetron, and any other 5-HT antagonist containing an imidazole, oxazole, thiazole, pyrazole, 3-pyrroline, or pyrrolidine group. In still other embodiments of the present invention, the therapeutic agent or drug is a combination of a 5-HT agonist and a non-steroidal anti-inflammatory drug (NSAID). Suitable NSAIDs for use in the present invention include, without limitation, traditional NSAIDs such as aspirin (i.e., acetylsalicylic acid), ibuprofen, flurbiprofen, acetaminophen, diclofenac, diflunisal, etodolac, indomethacin, ketoprofen, ketorolac, naproxen, nabumetone, oxaprozin, piroxicam, sulindac, and tolmetin; selective cyclooxygenase inhibitors such as celecoxib, rofecoxib, and valdecoxib; and combinations thereof.

The term “therapeutically effective amount” refers to the amount of a 5-HT agonist that is capable of achieving a therapeutic effect in a subject in need thereof. For example, a therapeutically effective amount of a 5-HT agonist can be the amount that is capable of preventing or relieving one or more symptoms associated with a migraine or a cluster headache.

The term “bioavailability” refers to the rate and/or extent to which a drug is absorbed or becomes available to the treatment site in the body.

As used herein, the phrase “substantially complete conversion of the 5-HT agonist from its ionized to its un-ionized form” refers to greater than about 50% conversion of the 5-HT agonist from its ionized form into its un-ionized form. For example, the buffer system may favor at least about 50%, 55%, 60%, 65%, 70%, 75%, 80%, 85%, 90%, 95%, or 99% conversion of the 5-HT agonist from its ionized form into its un-ionized form. In some embodiments, the conversion occurs within about 10 minutes following administration.

The term “administering” refers to administration of the compositions of the present invention to the mucous membranes of the oral cavity (i.e., oral mucosa). Examples of suitable sites of administration within the oral mucosa include, without limitation, the mucous membranes of the floor of the mouth (sublingual mucosa), the cheeks (buccal mucosa), the gums (gingival mucosa), the roof of the mouth (palatal mucosa), the lining of the lips, and combinations thereof. Preferably, the compositions of the present invention are administered to the sublingual mucosa, buccal mucosa, or a combination thereof.

II. General

The present invention provides novel compositions for the delivery of a 5-HT agonist across the oral mucosa. In particular, the buffer system in the compositions of the present invention raises the pH of saliva to a pH greater than about 9.9, thereby facilitating the substantially complete conversion of the 5-HT agonist from its ionized to its un-ionized form. Furthermore, delivery of the 5-HT agonist across the oral mucosa advantageously bypasses hepatic first pass metabolism of the drug and avoids enzymatic degradation of the drug within the gastrointestinal tract. As a result, the 5-HT agonist reaches the systemic circulation in a substantially shorter period of time and at a substantially higher concentration than with traditional oral (e.g., tablet) administration. Methods for using the compositions of the present invention for treating migraines are also provided.

The present invention is based upon the surprising discovery that the addition of an oxide component such as magnesium oxide to the buffer system is extremely beneficial for (a) raising the pH of saliva to a pH of about 9.9 or more, irrespective of starting pH; (b) reducing the corrosivity of the carbonate component present in the buffer system; (c) serving as a secondary binding agent thereby eliminating the need for stearic acid; and (d) lowering the amount of the carbonate component needed to produce the desired pH. Without intending to be bound by any particular theory, it is believed that the oxide component (e.g., magnesium oxide and aluminum-oxide) acts as a cytoprotective agent, protecting cells against the high pH of carbonate and bicarbonate components present in the buffered compositions.

III. Description of the Embodiments

The present invention provides, in one aspect, a composition for delivery of a 5-HT agonist across the oral mucosa, the composition comprising:
(a) a 5-HT agonist or a pharmaceutically acceptable salt thereof;
(b) a carrier; and
(c) a ternary buffer system comprising a carbonate salt, a bicarbonate salt, and a metal oxide,
   wherein the ternary buffer system raises the pH of saliva to a pH greater than about 9.9 irrespective of the starting pH of saliva.

In one embodiment, the ternary buffer system raises the pH of saliva to a pH of from about 9.9 to about 11, e.g., about 9.9, 10.0, 10.1, 10.2, 10.3, 10.4, 10.5, 10.6, 10.7, 10.8, 10.9, 11.0, irrespective of the starting pH of saliva. In another embodiment, the 5-HT agonist is selected from the group consisting of sumatriptan, naratriptan, rizatriptan, eletriptan, almotriptan, zolmitriptan, frovatriptan, and combinations thereof. In some embodiments, sumatriptan is the preferred 5-HT agonist. In certain instances, the composition further comprises a 5-HT antagonist. In certain other instances, the composition further comprises a non-steroidal anti-inflammatory drug (NSAID). In another embodiment, the carbonate salt is selected from the group consisting of sodium carbonate, potassium carbonate, calcium carbonate, ammonium carbonate, and magnesium carbonate. In yet another embodiment, the bicarbonate salt is selected from the group consisting of sodium bicarbonate, potassium bicarbonate, calcium bicarbonate, ammonium bicarbonate, and magnesium bicarbonate. In still yet another embodiment, the metal oxide is amorphous magnesium oxide or aluminum oxide. In a preferred embodiment, the ternary buffer system comprises sodium carbonate, sodium bicarbonate, and amorphous magnesium oxide. In another preferred embodiment, the sodium bicarbonate is dessicant-coated sodium bicarbonate.

In another embodiment, the compositions of the present invention are in a dosage form selected from the group consisting of a lozenge, a chewing gum, a chewable tablet, and a dissolving tablet such as a slow-dissolving tablet or a quick-dissolving tablet. Preferably, the composition is a lozenge or a dissolving tablet. A description of lozenge, chewing gum, and quick-dissolving tablet compositions containing a 5-HT agonist is provided in the Examples below.

In a preferred embodiment, the 5-HT agonist is delivered across an oral mucosa selected from the group consisting of the sublingual mucosa, the buccal mucosa, and a combination thereof. Preferably, the composition is administered sublingually so that the 5-HT agonist is delivered across the sublingual mucosa.

In another embodiment, the carrier is typically a solid, semi-solid, or liquid such as a binder, a gum base, or combinations thereof. Suitable binders for use in the compositions of the present invention include, without limitation, sugar alcohols such as mannitol, sorbitol, and xylitol; sugars such as lactose, dextrose, sucrose, glucose, and powdered sugar; other substances such as inositol, molasses, maltodextrin, starch, cellulose, microcrystalline cellulose, polyvinylpyrrolidone, acacia gum, guar gum, tragacanth gum, alginate, extract of Irish moss, panwar gum, ghatti gum, mucilage of isapol husks, Veegum®, larch arabogalactan, gelatin, methylcellulose, ethylcellulose, carboxymethylcellulose, hydroxypropylmethylcellulose, polyacrylic acid (e.g., Carbopol), calcium silicate, calcium phosphate, dicalcium phosphate, calcium sulfate, kaolin, sodium chloride, polyethylene glycol; and combinations thereof. Suitable gum bases for use in the compositions of the present invention include, for example, materials selected from among the many water-insoluble and saliva-insoluble gum base materials known in the art. In certain instances, the gum base comprises at least one hydrophobic polymer and at least one hydrophilic polymer. Non-limiting examples of suitable hydrophobic and hydrophilic polymers for gum bases include both natural and synthetic polymers such as elastomers, rubbers, and combinations thereof. Examples of suitable natural polymers include, without limitation, substances of plant origin such as chicle, jelutong, gutta percha, crown gum, and combinations thereof. Examples of suitable synthetic polymers include elastomers such as butadiene-styrene copolymers, isobutylene and isoprene copolymers (e.g., “butyl rubber”), polyethylene, polyisobutylene, polyvinylester (e.g., polyvinyl acetate and polyvinyl acetate phthalate), and combinations thereof. In other instances, the gum base comprises a mixture of butyl rubber (i.e., isobutylene and isoprene copolymer), polyisobutylene, and optionally, polyvinylacetate (e.g., having a molecular weight of approximately 12,000).

In yet another embodiment, the compositions of the present invention can further comprise a sweetening agent, a flavoring agent, a protecting agent, a plasticizer, a wax, an elastomeric solvent, a filler material, a preservative, or combinations thereof. In still yet another embodiment, the compositions of the present invention can further comprise a lubricating agent, a wetting agent, an emulsifying agent, a solubilizing agent, a suspending agent, a coloring agent, a disintegrating agent, or combinations thereof. In a preferred embodiment, the average particle size of the drug in the compositions described herein is about 20 microns, as compared to a typical average drug particle size of from about 75 to about 100 microns. In another preferred embodiment, the average particle size of the drug in the compositions described herein is less than or equal to the average particle size of the carrier ingredients (e.g., gum base, binders, etc.).

In preferred embodiments of the present invention, the 5-HT agonist is sumatriptan and the ternary buffer system comprises sodium carbonate, sodium bicarbonate, and amorphous magnesium oxide. Such compositions are preferably formulated in the form of a lozenge, candy, or dissolving tablet for sublingual administration. As a result, upon sublingual administration, sumatriptan is delivered across the sublingual mucosa. Preferably, the sodium bicarbonate is dessicant-coated sodium bicarbonate. In certain instances, a weight percent of amorphous magnesium oxide greater than or equal to the combined or individual weight percent of sodium carbonate and sodium bicarbonate is preferred. In certain other instances, a weight percent of amorphous magnesium oxide less than the combined or individual weight percent of sodium carbonate and sodium bicarbonate is used, e.g., from about 0.1% to about 10%.

In certain instances, the composition comprises from about 2.5 to about 4.5 weight percent sumatriptan; from about 4.0 to about 7.0 weight percent sodium carbonate; from about 8.0 to about 12.0 weight percent dessicant-coated sodium bicarbonate; and from about 20 to about 30 weight percent amorphous magnesium oxide. In a preferred embodiment, the composition comprises about 3.5 weight percent sumatriptan; about 5.5 weight percent sodium carbonate; about 9.0 weight percent dessicant-coated sodium bicarbonate; and about 25 weight percent amorphous magnesium oxide.

In another aspect, the present invention provides a composition for delivery of a 5-HT agonist across the oral mucosa, the composition comprising:
(a) a 5-HT agonist or a pharmaceutically acceptable salt thereof;
(b) a carrier; and
(c) a ternary buffer system comprising a carbonate salt, a bicarbonate salt, and a citrate, phosphate, or borate salt,
   wherein the ternary buffer system raises the pH of saliva to a pH greater than about 9.9 irrespective of the starting pH of saliva.

In one embodiment, the ternary buffer system raises the pH of saliva to a pH of from about 9.9 to about 11, irrespective of the starting pH of saliva. Suitable 5-HT agonists for use in the present invention are described above. Suitable carbonate salts and bicarbonate salts for use in the ternary buffer systems of the present invention are also described above. In certain instances, the composition further comprises a 5-HT antagonist. In certain other instances, the composition further comprises an NSAID.

Suitable citrate, phosphate, and borate salts include, without limitation, any salt of citric acid, phosphoric acid, or boric acid known in the art. For example, in some embodiments, the citrate salt is selected from the group consisting of sodium citrate, potassium citrate, calcium citrate, magnesium citrate, and ammonium citrate. In other embodiments, the phosphate salt is selected from the group consisting of monobasic sodium phosphate, dibasic sodium phosphate, monobasic potassium phosphate, dibasic potassium phosphate, monobasic calcium phosphate, dibasic calcium phosphate, monobasic magnesium phosphate, dibasic magnesium phosphate, monobasic ammonium phosphate, and dibasic ammonium phosphate. In yet other embodiments, the borate salt is selected from the group consisting of sodium borate, potassium borate, calcium borate, magnesium borate, and ammonium borate. In certain instances, the ternary buffer system comprises a carbonate salt, a bicarbonate salt, and a citrate salt. In certain other instances, the ternary buffer system comprises a carbonate salt, a bicarbonate salt, and a phosphate salt. In further instances, the ternary buffer system comprises a carbonate salt, a bicarbonate salt, and a borate salt.

In another embodiment, the compositions of the present invention are in any of the dosage forms described above. Preferably, the 5-HT agonist is delivered across an oral mucosa as described above. In yet another embodiment, the carrier is selected from the group consisting of a binder, a gum base, and combinations thereof. Suitable binders and gum bases for use in the compositions of the present invention are described above.

In still yet another embodiment, the compositions of the present invention can further comprise one or more of the additional agents described above. In preferred embodiments, the average particle size of the drug in the compositions described herein is about 20 microns and/or is less than or equal to the average particle size of the carrier ingredients (e.g. gum base, binders, etc.).

In preferred embodiments of the present invention, the 5-HT agonist is sumatriptan and the ternary buffer system comprises sodium carbonate, sodium bicarbonate, and a citrate, phosphate, or borate salt. Such compositions are preferably formulated in the form of a lozenge, candy, or dissolving tablet for sublingual administration.

In yet another aspect, the present invention provides a composition for delivery of a 5-HT agonist across the oral mucosa, the composition comprising:
(a) a 5-HT agonist or a pharmaceutically acceptable salt thereof;
(b) a carrier; and
(c) a buffer system comprising a carbonate salt or a bicarbonate salt and two or more buffering agents selected from the group consisting of a metal oxide, a citrate salt, a phosphate salt, and a borate salt,
   wherein the buffer system raises the pH of saliva to a pH greater than about 9.9 irrespective of the starting pH of saliva.

In one embodiment, the buffer system raises the pH of saliva to a pH of from about 9.9 to about 11, irrespective of the starting pH of saliva. Suitable 5-HT agonists for use in the present invention are described above. Suitable carbonate salts and bicarbonate salts for use in the buffer systems of the present invention are also described above. In certain instances, the composition further comprises a 5-HT antagonist. In certain other instances, the composition further comprises an NSAID.

In another embodiment, the metal oxide is magnesium oxide or aluminum oxide. Preferably, the magnesium oxide is amorphous magnesium oxide. Suitable citrate, phosphate, and borate salts include, without limitation, any salt of citric acid, phosphoric acid, or boric acid known in the art such as those described above. In certain instances, the buffer system comprises a carbonate salt or a bicarbonate salt, a metal oxide, and a citrate, phosphate, or borate salt. In certain other instances, the buffer system comprises a carbonate salt or a bicarbonate salt, a citrate salt, and a phosphate salt. In certain instances, the buffer system comprises a carbonate salt or a bicarbonate salt, a citrate salt, and a borate salt. In certain other instances, the buffer system comprises a carbonate salt or a bicarbonate salt, a phosphate salt, and a borate salt.

In yet another embodiment, the compositions of the present invention are in any of the dosage forms described above. Preferably, the 5-HT agonist is delivered across an oral mucosa as described above. In still yet another embodiment, the carrier is selected from the group consisting of a binder, a gum base, and combinations thereof. Suitable binders and gum bases for use in the compositions of the present invention are described above.

In a further embodiment, the compositions of the present invention can further comprise one or more of the additional agents described above. In preferred embodiments, the average particle size of the drug in the compositions described herein is about 20 microns and/or is less than or equal to the average particle size of the carrier ingredients (e.g., gum base, binders, etc.).

In preferred embodiments of the present invention, the 5-HT agonist is sumatriptan and the buffer system comprises sodium carbonate or sodium bicarbonate and two or more buffering agents selected from the group consisting of a metal oxide, a citrate salt, a phosphate salt, and a borate salt. Such compositions are preferably formulated in the form of a lozenge, candy, or dissolving tablet for sublingual administration.

In still yet another aspect, the present invention provides a composition for delivery of a 5-HT agonist across the oral mucosa, the composition comprising:
(a) a 5-HT agonist or a pharmaceutically acceptable salt thereof;
(b) a carrier; and
(c) a binary buffer system comprising a carbonate salt or a bicarbonate salt and a metal oxide,
   wherein the binary buffer system raises the pH of saliva to a pH greater than about 9.9 irrespective of the starting pH of saliva.

In one embodiment, the binary buffer system raises the pH of saliva to a pH of from about 9.9 to about 11, irrespective of the starting pH of saliva. Suitable 5-HT agonists for use in the present invention are described above. Suitable carbonate salts, bicarbonate salts, and metal oxides for use in the binary buffer systems of the present invention are also described above. In certain instances, the composition further comprises a 5-HT antagonist. In certain other instances, the composition further comprises an NSAID.

In another embodiment, the compositions of the present invention are in any of the dosage forms described above. Preferably, the 5-HT agonist is delivered across an oral mucosa as described above. In yet another embodiment, the carrier is selected from the group consisting of a binder, a gum base, and combinations thereof. Suitable binders and gum bases for use in the compositions of the present invention are described above.

In still yet another embodiment, the compositions of the present invention can further comprise one or more of the additional agents described above. In preferred embodiments, the average particle size of the drug in the compositions described herein is about 20 microns and/or is less than or equal to the average particle size of the carrier ingredients (e.g., gum base, binders, etc.).

In preferred embodiments of the present invention, the 5-HT agonist is sumatriptan and the binary buffer system comprises sodium carbonate or sodium bicarbonate and amorphous magnesium oxide. Such compositions are preferably formulated in the form of a lozenge, candy, or dissolving tablet for sublingual administration. In certain instances, a weight percent of amorphous magnesium oxide greater than or equal to the weight percent of sodium carbonate or sodium bicarbonate is preferred. In certain other instances, a weight percent of amorphous magnesium oxide less than the weight percent of sodium carbonate or sodium bicarbonate is used, e.g., from about 0.1% to about 10%.

In a further aspect, the present invention provides a composition for delivery of a 5-HT agonist across the oral mucosa, the composition comprising:
(a) a 5-HT agonist or a pharmaceutically acceptable salt thereof;
(b) a carrier; and
(c) a binary buffer system comprising a carbonate salt or a bicarbonate salt and a citrate, phosphate, or borate salt,
   wherein the binary buffer system raises the pH of saliva to a pH greater than about 9.9 irrespective of the starting pH of saliva.

In one embodiment, the binary buffer system raises the pH of saliva to a pH of from about 9.9 to about 11, irrespective of the starting pH of saliva. Suitable 5-HT agonists for use in the present invention are described above. Suitable carbonate salts, bicarbonate salts, and citrate, phosphate, and borate salts for use in the binary buffer systems of the present invention are also described above. In certain instances, the composition further comprises a 5-HT antagonist. In certain other instances, the composition further comprises an NSAID.

In another embodiment, the compositions of the present invention are in any of the dosage forms described above. Preferably, the 5-HT agonist is delivered across an oral mucosa as described above. In yet another embodiment, the carrier is selected from the group consisting of a binder, a gum base, and combinations thereof. Suitable binders and gum bases for use in the compositions of the present invention are described above.

In still yet another embodiment, the compositions of the present invention can further comprise one or more of the additional agents described above. In preferred embodiments, the average particle size of the drug in the compositions described herein is about 20 microns and/or is less than or equal to the average particle size of the carrier ingredients (e.g., gum base, binders, etc.).

In preferred embodiments of the present invention, the 5-HT agonist is sumatriptan and the binary buffer system comprises sodium carbonate or sodium bicarbonate and a citrate, phosphate, or borate salt. Such compositions are preferably formulated in the form of a lozenge, candy, or dissolving tablet for sublingual administration.

In another aspect, the present invention provides a composition for delivery of a 5-HT agonist across the oral mucosa, the composition comprising:
(a) a 5-HT agonist or a pharmaceutically acceptable salt thereof;
(b) a carrier; and
(c) a binary buffer system comprising a metal oxide and a citrate, phosphate, or borate salt,
   wherein the binary buffer system raises the pH of saliva to a pH greater than about 9.9 irrespective of the starting pH of saliva.

In one embodiment, the binary buffer system raises the pH of saliva to a pH of from about 9.9 to about 11, irrespective of the starting pH of saliva. Suitable 5-HT agonists for use in the present invention are described above. Suitable metal oxides and citrate, phosphate, and borate salts for use in the binary buffer systems of the present invention are also described above. In certain instances, the composition further comprises a 5-HT antagonist. In certain other instances, the composition further comprises an NSAID.

In another embodiment, the compositions of the present invention are in any of the dosage forms described above. Preferably, the 5-HT agonist is delivered across an oral mucosa as described above. In yet another embodiment, the carrier is selected from the group consisting of a binder, a gum base, and combinations thereof. Suitable binders and gum bases for use in the compositions of the present invention are described above.

In still yet another embodiment, the compositions of the present invention can further comprise one or more of the additional agents described above. In preferred embodiments, the average particle size of the drug in the compositions described herein is about 20 microns and/or is less than or equal to the average particle size of the carrier ingredients (e.g., gum base, binders, etc.).

In preferred embodiments of the present invention, the 5-HT agonist is sumatriptan and the binary buffer system comprises amorphous magnesium oxide and a citrate, phosphate, or borate salt. Such compositions are preferably formulated in the form of a lozenge, candy, or dissolving tablet for sublingual administration.

In yet another aspect, the present invention provides a method for treating a migraine in a subject in need thereof, the method comprising:

administering to the subject a composition comprising a therapeutically effective amount of a 5-HT agonist or a pharmaceutically acceptable salt thereof, a carrier, and a ternary buffer system comprising a carbonate salt, a bicarbonate salt, and a metal oxide, wherein the ternary buffer system raises the pH of saliva to a pH greater than about 9.9 irrespective of the starting pH of saliva.

In a preferred embodiment, the composition delivers the 5-HT agonist across the oral mucosa such as, for example, the sublingual mucosa, the buccal mucosa, or a combination thereof. Preferably, the composition is administered sublingually so that the 5-HT agonist is delivered across the sublingual mucosa. Suitable migraines that can be treated with the compositions of the present invention include, without limitation, a migraine without aura and a migraine with aura.

In one embodiment, the ternary buffer system raises the pH of saliva to a pH of from about 9.9 to about 11, irrespective of the starting pH of saliva. Suitable 5-HT agonists for use in the present invention are described above. Suitable carbonate salts, bicarbonate salts, and metal oxides for use in the ternary buffer systems of the present invention are also described above. In certain instances, the composition further comprises a 5-HT antagonist. In certain other instances, the composition further comprises an NSAID.

In addition to a ternary buffer system comprising a carbonate salt, a bicarbonate salt, and a metal oxide, other buffer systems are suitable for use in the compositions of the present invention. For example, in an alternative embodiment, the ternary buffer system comprises a carbonate salt, a bicarbonate salt, and a citrate, phosphate, or borate salt. In another alternative embodiment, the buffer system comprises a carbonate salt or a bicarbonate salt and two or more buffering agents selected from the group consisting of a metal oxide, a citrate salt, a phosphate salt, and a borate salt. In yet another alternative embodiment, the buffer system is a binary buffer system comprising a carbonate salt or a bicarbonate salt and a metal oxide. In still yet another alternative embodiment, the buffer system is a binary buffer system comprising, a carbonate salt or a bicarbonate salt and a citrate, phosphate, or borate salt. In a further alternative embodiment, the buffer system is a binary buffer system comprising a metal oxide and a citrate, phosphate, or borate salt. In still yet another alternative embodiment, the buffer system is a binary buffer system comprising a carbonate salt and a bicarbonate salt, preferably sodium carbonate and sodium bicarbonate.

In another embodiment, the compositions of the present invention are in any of the dosage forms described above. In yet another embodiment, the carrier is selected from the group consisting of a binder, a gum base, and combinations thereof. Suitable binders and gum bases for use in the compositions of the present invention are described above.

In still yet another embodiment, the compositions of the present invention can further comprise one or more of the additional agents described above. In preferred embodiments, the average particle size of the drug in the compositions described herein is about 20 microns and/or is less than or equal to the average particle size of the carrier ingredients (e.g., gum base, binders, etc.).

In preferred embodiments of the present invention, the 5-HT agonist is sumatriptan and the ternary buffer system comprises sodium carbonate, sodium bicarbonate, and amorphous magnesium oxide. Such compositions are preferably formulated in the form of a lozenge, candy, or dissolving tablet for sublingual administration. In certain instances, a weight percent of amorphous magnesium oxide greater than or equal to the combined or individual weight percent of sodium carbonate and sodium bicarbonate is preferred. In certain other instances, a weight percent of amorphous magnesium oxide less than the combined or individual weight percent of sodium carbonate and sodium bicarbonate is used, e.g., from about 0.1% to about 10%.

A. 5-HT Agonists

The 5-hydroxytryptamine (5-HT) agonists of the present invention are preferably selected from sumatriptan, naratriptan, rizatriptan, eletriptan, almotriptan, zolmitriptan, frovatriptan, F 11356, pharmaceutically acceptable salts thereof, and combinations thereof. More preferably, the 5-HT agonist is sumatriptan, in all suitable forms. The 5-HT agonists described herein are basic compounds with selective or non-selective vasoactivity on blood vessels.

In general, the 5-HT agonists of the present invention have an ionized form and an un-ionized form. In certain instances, the 5-HT agonist is initially present at least partly in an ionized form. In certain other instances, the 5-HT agonist is initially present in an un-ionized form. As described in more detail below, the buffer system of the compositions described herein helps to convert substantially all of the 5-HT agonist from its ionized form to its un-ionized form. Alternatively, the buffer system helps ensure that the 5-HT agonist, initially in an un-ionized form, remains in an un-ionized form.

As used herein, the term “5-HT agonist” includes all pharmaceutically acceptable forms of the 5-HT agonist being described. For example, the 5-HT agonist can be in a racemic or isomeric mixture, a solid complex bound to an ion exchange resin, or the like. In addition, the 5-HT agonist can be in a solvated form. The term “5-HT agonist” is also intended to include all pharmaceutically acceptable salts, derivatives, and analogs of the 5-HT agonist being described, as well as combinations thereof. For example, the pharmaceutically acceptable salts of the 5-HT agonist include, without limitation, the succinate, tartarate, bitartarate, hemisuccinate, citrate, maleate, hydrochloride, carbamate, sulfate, nitrate, and benzoate salt forms thereof, as well as combinations thereof and the like.

Conversion of the ionized form to the un-ionized form for the 5-HT agonist is related to pH according to the formula: pH=pKa+Log₁₀ (un-ionized concentration/ionized concentration). When the pH is the same as the pKa, equimolar concentrations of the un-ionized form and ionized form exist. For basic compounds such as the 5-HT agonists described herein, when the pH is one unit higher than the pKa, the ratio of the un-ionized form to the ionized form is 91:9. Similarly, when the pH is two units higher than the pKa, the ratio of un-ionized form to the ionized form is 100:1. As noted above, the un-ionized form is lipophilic and, therefore, more capable of passing through mucous membranes such as the oral mucosa than the ionized form, which is lipophobic in nature. Accordingly, increasing the pH of the saliva favors conversion of the ionized form into the un-ionized form for basic compounds such as the 5-HT agonists described herein, and the final pH can be determined by making use of the above formula.

The 5-HT agonists of the present invention are indole derivatives useful in the treatment of conditions such as migraines, having the following basic indole nucleus: 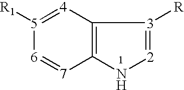
wherein R is typically an alkyl, alkenyl, cycloalkyl, or cycloalkenyl group and R_{I} is typically a sulfonamide, an oxazolidinone, a triazole, or a sulfonyl group, any of which may be optionally substituted. More particularly, the 5-HT agonists of the present invention are preferably selected from the group consisting of sumatriptan, naratriptan, rizatriptan, eletriptan, almotriptan, zolmitriptan, frovatriptan, and F 11356, having the following structures:

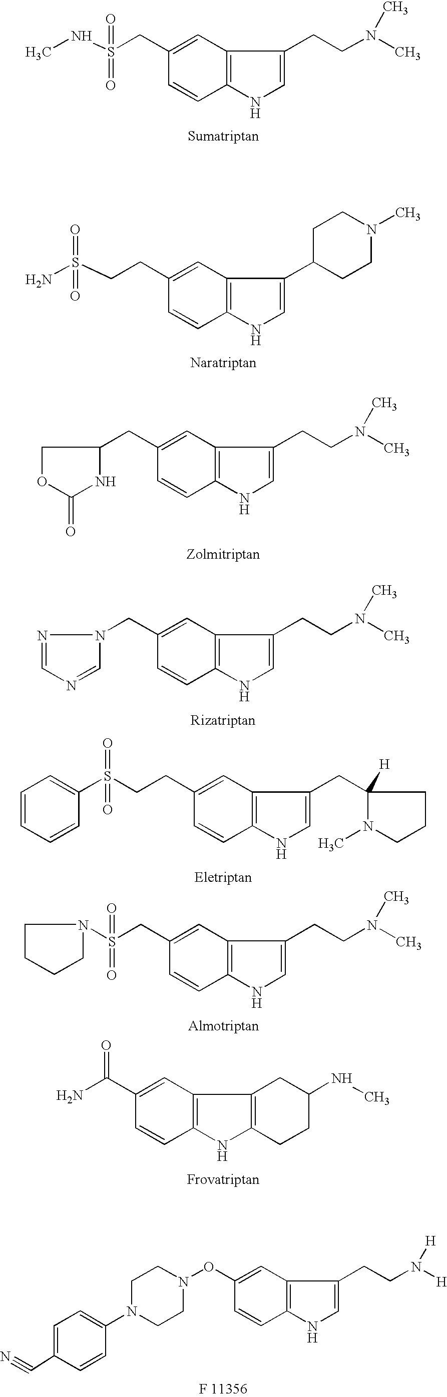

For the above-described 5-HT agonists, the primary, secondary, or tertiary amines typically control the extent of ionization of the compound.

The 5-HT agonists of the present invention bind with high affinity to one or more 5-HT₁ receptor subtypes. Without being bound to any particular theory, the therapeutic activity of the 5-HT agonists of the present invention in treating migraines is attributed to one or more of the following mechanisms: (1) activation of 5-HT₁ receptors located on intracranial blood vessels (e.g., arteriovenous anastomoses) by 5-HT agonists to stimulate vasoconstriction; and (2) activation of 5-HT₁. receptors located on sensory nerve endings in the trigeminal system to inhibit pro-inflammatory neuropeptide (e.g. vasoactive intestinal peptide, substance P, calcitonin gene-related peptide) release.

In other embodiments of the present invention, a 5-HT antagonist and/or a non-steroidal anti-inflammatory drug (NSAID) is delivered in combination with a 5-HT agonist. 5-HT antagonists typically consist of three main components: (1) an aromatic structure; (2) a carbonyl-containing linking moiety; and (3) an out-of-plane basic nitrogen containing heterocyclic group. These groups have the specific spatial arrangement shown below:

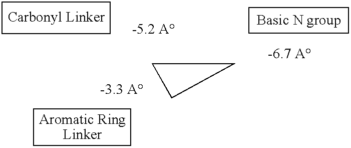

Suitable 5-HT antagonists for use in the present invention include, without limitation, 5-HT antagonists wherein the carbonyl linker is incorporated within the fused ring of the aromatic group (see, Table 1) and 5-HT antagonists wherein the carbonyl linker is directly attached (i.e., as a spacer unit) to the aromatic ring and the basic nitrogen group (see, Table 2).

TABLE 1
5-HT antagonists wherein the carbonyl linker is incorporated
within the fused aromatic ring.

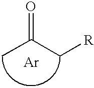

| 5-HT Antagonist | Ar | R |
|---|---|---|
| Ondansetron | 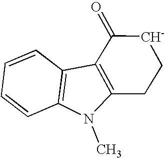 | 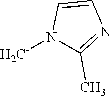 |
| Cilasetron | 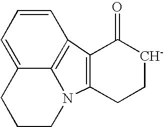 | 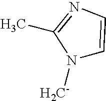 |
| Alosetron | 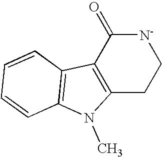 | 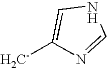 |
| Palonosetron | 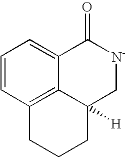 | 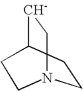 |

TABLE 2
5-HT antagonists wherein the carbonyl linker is attached to the
aromatic ring and the basic nitrogen group as a spacer.

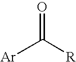

| 5-HT | | |
|---|---|---|
| Antagonist | Ar | R |
| Granisetron | 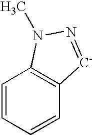 | 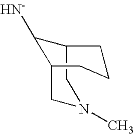 |
| Tropisetron | 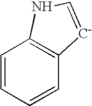 | 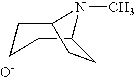 |
| Dolasetron | 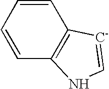 | 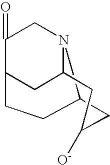 |
| Bernesetron | 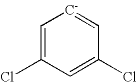 | 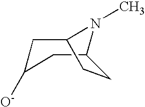 |
| Ramosetron | 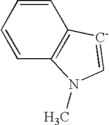 | 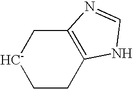 |
| Azasetron | 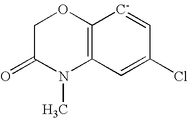 | 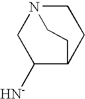 |
| Itasetron | 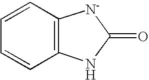 | 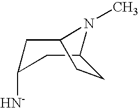 |
| Zacopride | 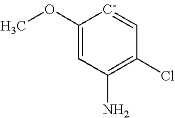 | 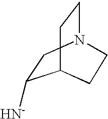 |

As shown in Tables 1 and 2, the constant feature among the 5-HT antagonists is the basic nitrogen group. The basic nitrogen group can be classified generally as an imidazole group or as a nitrogen-containing heterobicyclic derivative.

Using the above formula, the overall lipophilicity and ionization of the 5-HT antagonists can be controlled and modulated by regulating the pH of the medium containing the 5-HT antagonist relative to the pKa of the basic nitrogen group. For example, 5-HT antagonists that contain nitrogen in an imidazole group have a pKa in the region of about 7.4, and can thus be substantially converted to their un-ionized, lipophilic form at a pH greater than about 8.4. Similarly, 5-HT antagonists that contain nitrogen in a bicyclic ring have a pKa of about 8.8, and can thus be substantially converted to their un-ionized, lipophilic form at a pH greater than about 9.8. Specific examples of suitable 5-HT antagonists for use in the present invention include, without limitation, ondansetron, palonosetron, tropisetron, lerisetron, alosetron, granisetron, dolasetron, bemesetron, ramosetron, azaseteron, itasetron, zacopride, cilasetron, and any other 5-HT antagonist containing an imidazole, oxazole, thiazole, pyrazole, 3-pyrroline, or pyrrolidine group.

Suitable NSAIDs for use in the present invention include, without limitation, traditional NSAIDs such as aspirin (i.e., acetylsalicylic acid), ibuprofen, flurbiprofen, acetaminophen, diclofenac, diflunisal, etodolac, indomethacin, ketoprofen, ketorolac, naproxen, nabumetone, oxaprozin, piroxicam, sulindac, and tolmetin; selective cyclooxygenase inhibitors such as celecoxib, rofecoxib, and valdecoxib; and combinations thereof

B. Buffer Systems

Although a binary composition of sodium carbonate and sodium bicarbonate can generally raise the pH of saliva to a level of about 8.0-9.8, the carbonate component must be present in an amount substantially higher than the bicarbonate component when a pH of about 9.0-9.8 is desired. However, with higher levels of carbonate, a corrosive effect on the oral mucosa and other oral tissues generally develops. As such, binary compositions containing only sodium carbonate and sodium bicarbonate have reduced utility for delivering the therapeutic agents of the present invention across the oral mucosa.

The present invention overcomes such limitations by providing, in one embodiment, ternary buffer systems comprising a carbonate salt, a bicarbonate salt, and an oxide component such as, for example, magnesium oxide or aluminum oxide. Although basic buffering agents are typically used in the buffer systems of the present invention, one skilled in the art will appreciate that acidic agents can also be used to adjust the pH of the buffer system as long as the buffer system as a whole raises the pH of saliva to a pH greater than about 9.9 (e.g., about 9.9-11).

The concentration of each buffer system component is tailored such that the final salivary pH is achieved and sustained for a period of time, e.g., for at least about 2 minutes, at least about 5 minutes, at least about 10 minutes, at least about 20 minutes, or at least about 60 minutes. This typically involves a sensory and safety procedure involving adjusting the amounts of each buffer system component and measuring the final pH over time. In this way, selection of an appropriate weight ratio for each buffer system component can be easily determined in just a few trials. For example, the weight ratio of carbonate salt to bicarbonate salt for a ternary buffer system can be from about 1:10 to about 10:1, preferably from about 1:5 to about 5:1, more preferably from about 1:3 to about 3:1, and still more preferably from about 1:2 to about 2:1.

The carbonate salt is generally selected from sodium carbonate, potassium carbonate, calcium carbonate, ammonium carbonate, and magnesium carbonate. Preferably, the carbonate salt is sodium carbonate or potassium carbonate. Most preferably, the carbonate salt is sodium carbonate. Similarly, the bicarbonate salt is generally selected from sodium bicarbonate, potassium bicarbonate, calcium bicarbonate, ammonium bicarbonate, and magnesium bicarbonate. Preferably, the bicarbonate salt is sodium bicarbonate or potassium bicarbonate. Most preferably, the bicarbonate salt is sodium bicarbonate. In some embodiments, a dessicant-coated sodium bicarbonate is preferred. The amount of carbonate salt and bicarbonate salt used in the ternary buffer system is an amount that is sufficient, when used with the metal oxide, to raise salivary pH to a pH of about 9.5, preferably about 9.9 or more, and more preferably from about 9.9 to about 11, e.g. about 9.9, 10.0, 10.1, 10.2, 10.3, 10.4, 10.5, 10.6, 10.7, 10.8, 10.9, 11.0, irrespective of the starting pH.

In certain instances, the amount of bicarbonate salt in the ternary buffer system is greater than or equal to the amount of carbonate salt. For example, the weight ratio of bicarbonate salt to carbonate salt can be from about 1:1 to about 10:1, preferably from about 1:1 to about 5:1, and more preferably from about 1:1 to about 3:1. In a particularly preferred embodiment, the weight ratio of bicarbonate salt to carbonate salt is from about 1.5:1 to about 2:1. In certain other instances, the amount of bicarbonate salt in the ternary buffer system is less than the amount of carbonate salt.

Quite surprisingly, the addition of an oxide component such as magnesium oxide as a third component of the ternary buffer system has now been found to be extremely beneficial for (a) raising the pH of saliva to a pH of about 9.9 or more, irrespective of starting pH; (b) reducing the corrosivity of the carbonate component; (c) serving as a secondary binding agent thereby eliminating the need for stearic acid; and (d) lowering the amount of the carbonate component needed to produce the desired pH. Without intending to be bound by any particular theory, it is believed that the oxide component (e.g., magnesium oxide and aluminum oxide) acts as a cytoprotective agent, protecting cells against the high pH of carbonate and bicarbonate in the buffered compositions.

The amount of the oxide component used in the ternary buffer systems of the present compositions is an amount that is sufficient, when used with the remaining components, to raise salivary pH to a pH of about 9.5, preferably about 9.9 or more, and more preferably from about 9.9 to about 11, irrespective of the starting pH. In some embodiments, amorphous magnesium oxide is preferred. In certain instances, the weight percent of the oxide component is greater than or equal to the combined weight percent of the carbonate salt and the bicarbonate salt. For example, the weight ratio of the oxide component to the carbonate salt and the bicarbonate salt can be from about 1:1 to about 10:1, preferably from about 1:1 to about 5:1, and more preferably from about 1:1 to about 3: 1, and most preferably from about 1.5:1 to about 2:1. In certain other instances, the weight percent of the oxide component is greater than or equal to the weight percent of either the carbonate salt or the bicarbonate salt. In still other instances, the weight percent of the oxide component is less than the combined or individual weight percent of the carbonate salt and the bicarbonate salt, yet sufficient to provide an optimum pH of saliva as described above as well as good mouth feel properties.

In view of the above, the buffer systems of the present invention, in some of the most preferred embodiments, are ternary buffer systems containing sodium carbonate, sodium bicarbonate, and amorphous magnesium oxide.

Alternatively, in another embodiment, the buffer systems of the present invention are ternary buffer system comprising a carbonate salt, a bicarbonate salt, and a third buffering agent such as a citrate, phosphate, or borate salt. The concentration of each buffer system component is tailored such that the final salivary pH is achieved and sustained for a period of time, e.g., for at least about 2 minutes, at least about 5 minutes, at least about 10 minutes, at least about 20 minutes, or at least about 60 minutes.

Suitable carbonate salts and bicarbonate salts are described above. The amount of carbonate salt and bicarbonate salt used in the ternary buffer system is an amount that is sufficient, when used with the third buffering agent, to raise salivary pH to a pH of about 9.5, preferably about 9.9 or more, and more preferably from about 9.9 to about 11, irrespective of the starting pH.

The third buffering agent is generally selected from a citrate salt such as sodium citrate, potassium citrate, calcium citrate, magnesium citrate, and ammonium citrate; a phosphate salt such as monobasic sodium phosphate, dibasic sodium phosphate, monobasic potassium phosphate, dibasic potassium phosphate, monobasic calcium phosphate, dibasic calcium phosphate, monobasic magnesium phosphate, dibasic magnesium phosphate, monobasic ammonium phosphate, and dibasic ammonium phosphate; a borate salt such as sodium borate, potassium borate, calcium borate, magnesium borate, and ammonium borate; an ascorbate salt such as potassium ascorbate or sodium ascorbate; an acetate salt such as potassium acetate or sodium acetate; and alkaline starch. However, one skilled in the art will appreciate that essentially any salt of citric acid, phosphoric acid, boric acid, ascorbic acid, or acetic acid is suitable for use in the buffer systems of the present invention. The amount of the third buffering agent used in the ternary buffer system is an amount that is sufficient, when used with the remaining components, to raise salivary pH to a pH of about 9.5, preferably about 9.9 or more, and more preferably from about 9.9 to about 11, irrespective of the starting pH.

In certain instances, the amount of the carbonate salt or bicarbonate salt in the ternary buffer system is greater than or equal to the amount of the third buffering agent. For example, the weight ratio of the carbonate salt or bicarbonate salt to the third buffering agent can be from about 1:1 to about 10: 1, preferably from about 1:1 to about 5: 1, and more preferably from about 1:1 to about 3:1. In certain other instances, the amount of the carbonate salt or bicarbonate salt in the ternary buffer system is less than or equal to the amount of the third buffering agent. For example, the weight ratio of the carbonate salt or bicarbonate salt to the third buffering agent can be from about 1:1 to about 1:10, preferably from about 1:1 to about 1:5, and more preferably from about 1:1 to about 1:3.

Alternatively, in yet another embodiment, the buffer systems of the present invention are buffer systems comprising a carbonate salt or a bicarbonate salt and two or more buffering agents selected from the group consisting of a metal oxide, a citrate salt, a phosphate salt, and a borate salt. The concentration of each buffer system component is tailored such that the final salivary pH is achieved and sustained for a period of time, e.g., for at least about 2 minutes, at least about 5 minutes, at least about 10 minutes, at least about 20 minutes, or at least about 60 minutes.

Suitable carbonate salts and bicarbonate salts are described above. The amount of carbonate salt or bicarbonate salt used in the buffer system is an amount that is sufficient, when used with the remaining components, to raise salivary pH to a pH of about 9.5, preferably about 9.9 or more, and more preferably from about 9.9 to about 11, irrespective of the starting pH.

The two or more buffering agents are generally selected from a metal oxide, a citrate salt, a phosphate salt, a borate salt, an ascorbate salt, an acetate salt, and alkaline starch. Suitable metal oxides include, without limitation, magnesium oxide and aluminum oxide. Suitable citrate, phosphate, borate, ascorbate, and acetate salts include, without limitation, essentially any salt of citric acid, phosphoric acid, boric acid, ascorbic acid, or acetic acid known in the art such as those described above. The amount of the additional buffering agents used in the buffer system is an amount that is sufficient, when used with the carbonate salt or bicarbonate salt, to raise salivary pH to a pH of about 9.5, preferably about 9.9 or more, and more preferably from about 9.9 to about 11, irrespective of the starting pH.

Incertain instances, the buffer system comprises a carbonate salt or a bicarbonate, salt, a metal oxide, and a citrate, phosphate, or borate salt. Preferably, the metal oxide is amorphous magnesium oxide. In certain other instances, the buffer system comprises a carbonate salt or a bicarbonate salt, a citrate salt, and a phosphate salt. In certain instances, the buffer system comprises a carbonate salt or a bicarbonate salt, a citrate salt, and a borate salt. In certain other instances, the buffer system comprises a carbonate salt or a bicarbonate salt, a phosphate salt, and a borate salt.

In certain instances, the amount of the carbonate salt or bicarbonate salt in the buffer system is greater than or equal to the amount of the metal oxide or the citrate, phosphate, or borate salt. For example, the weight ratio of the carbonate salt or bicarbonate salt to the metal oxide or the citrate, phosphate, or borate salt can be from about 1:1 to about 10:1, preferably from about 1:1 to about 5:1, and more preferably from about 1:1 to about 3:1. In certain other instances, the amount of the carbonate salt or bicarbonate salt in the buffer system is less than or equal to the amount of the metal oxide or the citrate, phosphate, or borate salt. For example, the weight ratio of the carbonate salt or bicarbonate salt to the metal oxide or the citrate, phosphate, or borate salt can be from about 1:1 to about 1:10, preferably from about 1:1 to about 1:5, and more preferably from about 1:1 to about 1:3.

Alternatively, in still yet another embodiment, the buffer systems of the present invention are binary buffer systems comprising a carbonate salt or a bicarbonate salt and a metal oxide. The concentration of each buffer system component is tailored such that the final salivary pH is achieved and sustained for a period of time, e.g., for at least about 2 minutes, at least about 5 minutes, at least about 10 minutes, at least about 20 minutes, or at least about 60 minutes.

Suitable carbonate salts, bicarbonate salts, and metal oxides are described above. The amount of carbonate salt or bicarbonate salt used in the binary buffer system is an amount that is sufficient, when used with the oxide component, to raise salivary pH to a pH of about 9.5, preferably about 9.9 or more, and more preferably from about 9.9 to about 11, irrespective of the starting pH. In certain instances, the amount of the oxide component in the binary buffer system is greater than or equal to the amount of either the carbonate salt or bicarbonate salt. For example, the weight ratio of the oxide component to the carbonate salt or bicarbonate salt can be from about 1:1 to about 10:1, preferably from about 1:1 to about 5:1, and more preferably from about 1:1 to about 3:1. In certain other instances, the amount of the oxide component in the binary buffer system is less than the amount of either the carbonate salt or bicarbonate salt.

Alternatively, in a further embodiment, the buffer systems of the present invention are binary buffer systems comprising a carbonate salt or a bicarbonate salt and a second buffering agent such as a citrate, phosphate, or borate salt. The concentration of each buffer system component is tailored such that the final salivary pH is achieved and sustained for a period of time, e.g., for at least about 2 minutes, at least about 5 minutes, at least about 10 minutes, at least about 20 minutes, or at least about 60 minutes.

Suitable carbonate salts and bicarbonate salts are described above. The amount of carbonate salt or bicarbonate salt used in the binary buffer system is an amount that is sufficient, when used with the second buffering agent, to raise salivary pH to a pH of about 9.5, preferably about 9.9 or more, and more preferably from about 9.9 to about 11, irrespective of the starting pH. In certain instances, the amount of the second buffering agent in the binary buffer system is greater than or equal to the amount of either the carbonate salt or bicarbonate salt. For example, the weight ratio of the second buffering agent to the carbonate salt or bicarbonate salt can be from about 1:1 to about 10:1, preferably from about 1:1 to about 5:1, and more preferably from about 1:1 to about 3:1. In certain other instances, the amount of the second buffering agent in the binary buffer system is less than or equal to the amount of either the carbonate salt or bicarbonate salt. For example, the weight ratio of the second buffering agent to the carbonate salt or bicarbonate salt can be from about 1:1 to about 1:10, preferably from about 1:1 to about 1:5, and more preferably from about 1:1 to about 1:3.

The second buffering agent is generally selected from a citrate salt, a phosphate salt, a borate salt, an ascorbate salt, an acetate salt, and alkaline starch as described above. The amount of the second buffering agent used in the binary buffer system is an amount that is sufficient, when used with the carbonate salt or bicarbonate salt, to raise salivary pH to a pH of about 9.5, preferably about 9.9 or more, and more preferably from about 9.9 to about 11, irrespective of the starting pH.

Alternatively, in another embodiment, the buffer systems of the present invention are binary buffer systems comprising a metal oxide and a citrate, phosphate, or borate salt. The concentration of each buffer system component is tailored such that the final salivary pH is achieved and sustained for a period of time, e.g., for at least about 2 minutes, at least about 5 minutes, at least about 10 minutes, at least about 20 minutes, or at least about 60 minutes.

The metal oxide is typically magnesium oxide or aluminum oxide. Preferably, the magnesium oxide is amorphous magnesium oxide. Suitable citrate, phosphate, and borate salts include, without limitation, essentially any salt of citric acid, phosphoric acid, or boric acid known in the art such as those described above. The amount of the metal oxide used in the binary buffer system is an amount that is sufficient, when used with the citrate, phosphate, or borate salt, to raise salivary pH to a pH of about 9.5, preferably about 9.9 or more, and more preferably from about 9.9 to about 11, irrespective of the starting pH. Similarly, the amount of the citrate, phosphate, or borate salt used in the binary buffer system is an amount that is sufficient, when used with the metal oxide, to raise salivary pH to a pH of about 9.5, preferably about 9.9 or more, and more preferably from about 9.9 to about 11, irrespective of the starting pH.

In certain instances, the amount of the metal oxide in the binary buffer system is greater than or equal to the amount of the citrate, phosphate, or borate salt. For example, the weight ratio of the metal oxide to the citrate, phosphate, or borate salt can be from about 1:1 to about 10:1, preferably from about 1:1 to about 5:1, and more preferably from about 1:1 to about 3:1. In certain other instances, the amount of the metal oxide in the binary buffer system is less than or equal to the amount of the citrate, phosphate, or borate salt. For example, the weight ratio of the metal oxide to the citrate, phosphate, or borate salt can be from about 1:1 to about 1:10, preferably from about 1:1 to about 1:5, and more preferably from about 1:1 to about 1:3.

While the foregoing discussion has focused on the ability of the buffer system to alter salivary pH to favor substantial conversion to the unionized form of a therapeutic agent, the buffer system may also have subsidiary beneficial effects on the extent of absorption across the oral mucosa For example, the buffer system can create a final salivary pH that in turn affects the molecular configuration of the therapeutic agent in a way in which absorption across the oral mucosa is increased. It is to be understood that these subsidiary beneficial effects of the buffer system are still further advantages of the present invention and are within the general scope of the buffer system and compositions herein described.

C. Dosage Forms

The compositions of the present invention may take the form of solid, semi-solid, lyophilized powder, or liquid dosage forms, such as, for example, tablets (e.g., chewable, slow-dissolving, quick-dissolving), pills, capsules, lozenges, candies, gums, powders, solutions, suspensions, emulsions, aerosols, or the like. Preferably, the dosage form is a chewing gum, quick-dissolving tablet, candy, or lozenge.

While each subject or patient possesses unique factors that may affect the rate and extent of absorption of the therapeutic agents described herein, dosage forms such as chewing gums, quick-dissolving tablets, or lozenges offer advantages over the traditional dosage forms for oral administration. For example, each of these dosage forms avoids hepatic first pass metabolism, degradation within the gastrointestinal tract, and drug loss during absorption. Consequently, the amount of therapeutic agent required per dose is less than that which would be required if formulated, for example, in a pill or tablet for oral administration. Similarly, with each of these dosage forms, the bioavailability of the therapeutic agent is increased, thereby reducing the time to onset of therapeutic activity.

As used herein, the term “dosage form” refers to physically discrete units suitable as unitary dosages for human subjects and other mammals, each unit containing a predetermined quantity of therapeutic agent calculated to produce the desired onset, tolerability, and therapeutic effects, in association with one or more suitable pharmaceutical excipients such as carriers. Methods for preparing such dosage forms are known or will be apparent to those skilled in the art. For example, in some embodiments, a chewing gum dosage form of the present invention can be prepared according to the procedures set forth in U.S. Pat. No. 4,405,647. In other embodiments, a tablet, lozenge, or candy dosage form of the present invention can be prepared according to the procedures set forth in, for example, Remington: The Science and Practice of Pharmacy, 20^{th} Ed., Lippincott, Williams & Wilkins (2003); Pharmaceutical Dosage Forms, Volume 1: Tablets, 2^{nd} Ed., Marcel Dekker, Inc., New York, N.Y. (1989); and similar publications. The dosage form to be administered will, in any event, contain a quantity of the therapeutic agent in a therapeutically effective amount for relief of the condition being treated when administered in accordance with the teachings of this invention.

As used herein, the term “carrier” refers to a typically inert substance used as a diluent or vehicle for a drug such as a therapeutic agent. The term also encompasses a typically inert substance that imparts cohesive qualities to the composition. Suitable carriers for use in the compositions of the present invention include, without limitation, a solid, semi-solid, or liquid such as a binder or a gum base. Non-limiting examples of binders include mannitol, sorbitol, xylitol, maltodextrin, lactose, dextrose, sucrose, glucose, inositol, powdered sugar, molasses, starch, cellulose, microcrystalline cellulose, polyvinylpyrrolidone, acacia gum, guar gum, tragacanth gum, alginate, extract of Irish moss, panwar gum, ghatti gum, mucilage of isapol husks, Veegum®, larch arabogalactan, gelatin, methylcellulose, ethylcellulose, carboxymethylcellulose, hydroxypropylmethylcellulose, polyacrylic acid (e.g., Carbopol), calcium silicate, calcium phosphate, dicalcium phosphate, calcium sulfate, kaolin, sodium chloride, polyethylene glycol, and combinations thereof. These binders can be pre-processed to improve their flowability and taste by methods known in the art such as freeze drying (see, e.g., Fundamentals of Freeze-Drying, Pharm. Biotechnol., 14:281-360 (2002); Lyophililization of Unit Dose Pharmaceutical Dosage Forms, Drug. Dev. Ind. Pharm., 29:595-602 (2003)); solid-solution preparation (see, e.g., U.S. Pat. No. 6,264,987); and lubricant dusting and wet-granulation preparation with a suitable lubricating agent (see, e.g., Remington: The Science and Practice of Pharmacy, supra). For example, Mannogem® and Sorbogem®, sold by SPI Pharma Group (New Castle, Del.), are freeze-dried processed forms of mannitol and sorbitol, respectively. Typically, the compositions of the present invention comprise from about 25% to about 90% by weight of the binder, and preferably from about 50% to about 80%. However, one skilled in the art will appreciate that the compositions of the present invention can be made without any binders, e.g., to produce a highly friable dosage form.

Non-limiting examples of gum bases include materials selected from among the many water-insoluble and saliva-insoluble gum base materials known in the art. For example, in some instances, the gum base comprises at least one hydrophobic polymer and at least one hydrophilic polymer. Non-limiting examples of suitable hydrophobic and hydrophilic polymers for gum bases include both natural and synthetic polymers such as elastomers, rubbers, and combinations thereof. Examples of suitable natural polymers include, without limitation, substances of plant origin such as chicle, jelutong, gutta percha, crown gum, and combinations thereof. Examples of suitable synthetic polymers include elastomers such as butadiene-styrene copolymers, isobutylene and isoprene copolymers (e.g., “butyl rubber”), polyethylene, polyisobutylene, polyvinylester (e.g., polyvinyl acetate and polyvinyl acetate phthalate), and combinations thereof. In other instances, the gum base comprises a mixture of butyl rubber (i.e., isobutylene and isoprene copolymer), polyisobutylene, and optionally, polyvinylacetate (e.g., having a molecular weight of approximately 12,000). Typically, the gum base comprises from about 25% to about 75% by weight of these polymers, and preferably from about 30% to about 60%.

The compositions of the present invention can additionally include lubricating agents; wetting agents; emulsifying agents; solubilizing agents; suspending agents; preserving agents such as methyl-, ethyl-, and propyl-hydroxy-benzoates, butylated hydroxytoluene, and butylated hydroxyanisole; sweetening agents; flavoring agents; coloring agents; and disintegrating agents (i.e., dissolving agents) such as crospovidone as well as croscarmellose sodium and other cross-linked cellulose polymers.

Lubricating agents can be used to prevent adhesion of the dosage form to the surface of the dies and punches, and to reduce inter-particle friction. Lubricating agents may also facilitate ejection of the dosage form from the die cavity and improve the rate of granulation flow during processing. Examples of suitable lubricating agents include, without limitation, magnesium stearate, calcium stearate, zinc stearate, stearic acid, simethicone, silicon dioxide, talc, hydrogenated vegetable oil, polyethylene glycol, mineral oil, and combinations thereof. The compositions of the present invention can comprise from about 0% to about 10% by weight of the lubricating agent, and preferably from about 1% to about 5%.

Sweetening agents can be used to improve the palatability of the composition by masking any unpleasant tastes it may have. Examples of suitable sweetening agents include, without limitation, compounds selected from the saccharide family such as the mono-, di-, tri-, poly-, and oligosaccharides; sugars such as sucrose, glucose (corn syrup), dextrose, invert sugar, fructose, maltodextrin, and polydextrose; saccharin and salts thereof such as sodium and calcium salts; cyclamic acid and salts thereof; dipeptide sweeteners; chlorinated sugar derivatives such as sucralose and dihydrochalcone; sugar alcohols such as sorbitol, sorbitol syrup, mannitol, xylitol, hexa-resorcinol, and the like, and combinations thereof. Hydrogenated starch hydrolysate, and the potassium, calcium, and sodium salts of 3,6-dihydro-6-methyl-1-1,2,3-oxathiazin-4-one-2,2-dioxide may also be used. Of the foregoing, sorbitol, mannitol, and xylitol, either alone or in combination, are preferred sweetening agents. The compositions of the present invention can comprise from about 0% to about 80% by weight of the sweetening agent, preferably from about 5% to about 75%, and more preferably from about 25% to about 50%.

Flavoring agents can also be used to improve the palatability of the composition. Examples of suitable flavoring agents include, without limitation, natural and/or synthetic (i.e., artificial) compounds such as peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (e.g. white, milk, dark), vanilla, caramel, coffee, hazelnut, combinations thereof, and the like. Coloring agents can be used to color code the composition, for example, to indicate the type and dosage of the therapeutic agent therein. Suitable coloring agents include, without limitation, natural and/or artificial compounds such as FD & C coloring agents, natural juice concentrates, pigments such as titanium oxide, silicon dioxide, and zinc oxide, combinations thereof, and the like. The compositions of the present invention can comprise from about 0% to about 10% by weight of the flavoring and/or coloring agent, preferably from about 0.1% to about 5%, and more preferably from about 2% to about 3%.

1. Chewing Gums

When the dosage form is a chewing gum, the compositions of the present invention comprise a 5-HT agonist or a pharmaceutically acceptable salt thereof, a carrier such as a gum base, a binary or ternary buffer system, and optionally a protecting agent. The chewing gum composition may further comprise lubricating agents, wetting agents, emulsifying agents, suspending agents, preserving agents, sweetening agents, flavoring agents, and coloring agents. Typically, the chewing gum composition comprises from about 0.001% to about 10.0% by weight of the 5-HT agonist (in whatever chosen form, measured as per its free base form), more typically from about 0.01% to about 5.0%, and still more typically from about 0.1% to about 3.0%. One skilled in the art understands that the foregoing percentages will vary depending upon the particular source of 5-HT agonist utilized, the amount of 5-HT agonist desired in the final formulation, as well as on the particular release rate of 5-HT agonist desired. The binary or ternary buffer system of the chewing gum composition provides for a final salivary pH in excess of at least about 9.5, preferably at least about 9.9, and more preferably in the range of from about 9.9 to about 1. The chewing gum composition typically comprises from about 20% to about 95% by weight of the gum base, more typically from about 30% to about 85%, and most typically from about 50% to about 70% of the gum base.

The chewing gum composition may further comprise a protecting agent. The protecting agent coats at least part of the therapeutic agent, typically upon the mixing of the two agents. The protecting agent may be mixed with the therapeutic agent in a ratio of from about 0.1 to about 100 by weight, preferably in a ratio of from about 1 to about 50, and more preferably in a ratio of about 1 to about 10. Without being bound to any particular theory, the protecting agent reduces the adhesion between the therapeutic agent and the gum base so that the therapeutic agent may be more easily released from the gum base. In this way, the therapeutic agent may be delivered across the mucous membranes of the oral cavity within about 5 to about 20 minutes of chewing, preferably within about 10 minutes of chewing. A variety of different protecting agents may be used. Examples of suitable protecting agents include, without limitation, calcium stearate, glycerin monostearate, glyceryl behenate, glyceryl palmitostearate, hydrogenated castor oil, hydrogenated vegetable oil type I, light mineral oil, magnesium lauryl sulfate, magnesium stearate, mineral oil, poloxamer, polyethylene gycol, sodium benzoate, sodium chloride, sodium lauryl sulfate, stearic acid, cab-o-sil, talc, zinc stearate, and combinations thereof.

The gum base may additionally include plasticizers such as softeners or emulsifiers. Such plasticizers may, for example, help reduce the viscosity of the gum base to a desirable consistency and improve its overall texture and bite. Plasticizers may also facilitate the release of the therapeutic agent upon mastication. Non-limiting examples of plasticizers include lecithin, mono- and diglycerides, lanolin, stearic acid, sodium stearate, potassium stearate, glycerol triacetate, glycerol monostearate, glycerin, and combinations thereof. The gum base typically comprises from about 0% to about 20% by weight of the plasticizer, and more typically from about 5% to about 15%.

The gum base may further comprise waxes such as beeswax and microcrystalline wax, fats or oils such as soybean and cottonseed oil, and combinations thereof. Typically, the gum base comprises from about 0% to about 25% by weight of these waxes and oils, and more typically comprises from about 15% to about 20%.

In addition, the gum base may further comprise one or more elastomeric solvents such as rosins and resins. Non-limiting examples of such solvents include methyl, glycerol, and pentaerythritol esters of rosins, modified rosins such as hydrogenated, dimerized or polymerized rosins, or combinations thereof (e.g., pentaerythritol ester of partially hydrogenated wood rosin, pentaerythritol ester of wood rosin, glycerol ester of wood rosin, glycerol ester of partially dimerized rosin, glycerol ester of polymerized rosin, glycerol ester of tall oil rosin, glycerol ester of wood rosin and partially hydrogenated wood rosin and partially hydrogenated methyl ester of rosin such as polymers of alpha-pinene or beta-pinene, terpene resins including polyterpene, and combinations thereof). Typically, the gum base comprises from about 0% to about 75% by weight of the elastomeric solvent, and more typically less than about 10%.

The gum base may further comprise a filler material to enhance the chewability of the final chewing gum composition. Fillers that are substantially non-reactive with other components of the final chewing gum formulation are preferable. Examples of suitable fillers include, without limitation, calcium carbonate, magnesium silicate (i.e., talc), dicalcium phosphate, metallic mineral salts (e.g., alumina, aluminum hydroxide, and aluminum silicates), and combinations thereof. Typically, the gum base comprises from about 0% to about 30% by weight of the filler, and more typically from about 10% to about 20%.

One skilled in the art will appreciate that the gum base need not be prepared from its individual components. For example, the gum base can be purchased with the desired ingredients contained therein, and can be modified to include additional agents. Several manufacturers produce gum bases suitable for use with the described chewing gum compositions. Examples of such gum bases include, without limitation, Pharmgum™ M, S, or C (SPI Pharma Group; New Castle, Del.). In general, Pharmagum™ comprises a mixture of gum base, sweetening agent, plasticizer, and sugar.

In certain instances, the chewing gum composition includes a therapeutic agent centerfill. A centerfill may be particularly suitable when immediate release of the therapeutic agent is preferred. In addition, encapsulating the therapeutic agent in a centerfill may help to mask any undesirable taste that the therapeutic agent may have. In these instances, the gum base surrounds, at least in part, a centerfill. The centerfill comprises at least one therapeutic agent, and may be a liquid or semi-liquid material. The centerfill material can be a synthetic polymer, a semi-synthetic polymer, low-fat, or fat-free and contain one or more sweetening agents, flavoring agents, coloring agents, and/or scenting agents. Preferably, the centerfill includes a binary or ternary buffer system as described herein. Methods for preparing a centerfill chewing gum are described, for example, in U.S. Pat. No. 3,806,290, which is hereby incorporated by reference in its entirety.

The chewing gum compositions can have any desired shape, size, and texture. For example, the composition can have the shape of a stick, tab, gumball, and the like. Similarly, the chewing gum can be any desirable color. For example, the chewing gum can be any shade of red, blue, green, orange, yellow, violet, indigo, and mixtures thereof, and can be color coded to indicate the type and dosage of the therapeutic agent therein. The chewing gum can be individually wrapped or grouped together in pieces for packaging by methods well known in the art.

2. Tablets

When the dosage form is a tablet such as a dissolving tablet (i.e., disintegrating tablet) or chewable tablet, the compositions of the present invention comprise a 5-HT agonist or a pharmaceutically acceptable salt thereof, a carrier such as a binder, and a binary or ternary buffer system. The tablet composition may further comprise lubricating agents, wetting agents, emulsifying agents, suspending agents, preserving agents, sweetening agents, flavoring agents, coloring agents, and disintegrating agents. Typically, the tablet compositions of the present invention comprise from about 0.001% to about 10.0% by weight of the 5-HT agonist (in whatever chosen form, measured as per its free base form), and more typically from about 1.0% to about 5.0%. In some embodiments, about 3.5% by weight of the 5-HT agonist is used. One skilled in the art understands that the foregoing percentages will vary depending upon the particular source of 5-HT agonist utilized, the amount of 5-HT agonist desired in the final formulation, as well as on the particular release rate of 5-HT agonist desired. The buffer system of the tablet composition provides for a final salivary pH in excess of at least about 9.5, preferably at least about 9.9, and more preferably in the range of from about 9.9 to about 11.

In certain embodiments, the tablet is a dissolving tablet such as a slow-dissolving or quick-dissolving tablet that is dissolved by a subject's saliva, without the need for chewing. For example, a dissolving tablet placed on the subject's tongue can be used for buccal delivery of the therapeutic agent. Alternatively, a dissolving tablet placed underneath the subject's tongue can be used for sublingual delivery of the therapeutic agent. This type of dosage form may be particularly desirable for pediatric and geriatric patients, since small children and aged individuals often have difficulty chewing certain items. Typically, the dissolving tablet is formulated to dissolve within about 1 to about 15 minutes, preferably within about 2 to about 10 minutes, e.g., within about 2, 3, 4, 5, 6, 7, 8, 9, or 10 minutes, following administration. One skilled in the art will understand that quick-dissolving tablets dissolve faster than slow-dissolving tablets, which are typically dissolved gradually rather than rapidly by a subject's saliva. In a preferred embodiment, the slow-dissolving or quick-dissolving tablet delivers the therapeutic agent across the sublingual mucosa.

In certain other embodiments, the tablet is a chewable tablet that is chewed by a subject and formulated to dissolve either rapidly or gradually. For example, a chewable tablet placed on the subject's tongue can be used for buccal delivery of the therapeutic agent. During chewing, the chewable tablet can be moved around within the mouth and can sometimes be parked between the gums and the cheeks or underneath the tongue. As a result, at least a portion of the therapeutic agent contained within a chewable tablet may also be delivered sublingually (i.e., across the sublingual mucosa). Typically, the chewable tablet is formulated to dissolve within about 1 to about 15 minutes, preferably within about 2 to about 10 minutes, e.g., within about 2, 3, 4, 5, 6, 7, 8, 9, or 10 minutes, following administration.

As described above, the dissolving and chewable tablets of the present invention are typically formulated to dissolve within about 1 to 15 minutes following administration. However, while these time frames are amenable to maximum exposure of the therapeutic agent to the oral mucosa (e.g., to the sublingual and/or buccal mucosa), they are not always amenable to user compliance (e.g., users may swallow too frequently and, therefore, hinder maximal transmucosal absorption). Consequently, in certain instances, it may be desirable to strike a balance between patient compliance and maximum exposure time of the therapeutic agent to the oral mucosa. This can be accomplished, for example, by reducing the tablet size (e.g., from about 700-800 mg to about 200-300 mg) without reducing the concentration or amount per unit dose of the buffer system or the therapeutic agent. In addition, subtle changes to the tablet formulation such as, for example, replacing one flavoring agent for another (e.g., chocolate for spearmint) or replacing one binder or sweetening agent for another (e.g., lactose for mannitol or sorbitol) may be used to reduce salivation.

The carrier present in the tablets of the present invention is typically a binder that is useful in keeping the tablet in a semi-solid state, and may be a solid or a liquid, and may for example be a high-melting point fat or waxy material. Materials suitable as binders are discussed in detail above and may be used alone or in combination in the tablet compositions of the present invention. In addition, binders such as mannitol, sorbitol, lactose, sucrose, and inositol can impart properties to the tablet that permit or enhance its disintegration in the mouth.

The tablet composition may further comprise a protecting agent. The protecting agent coats at least part of the therapeutic agent, typically upon the mixing of the two agents. The protecting agent may be mixed with the therapeutic agent in a ratio of from about 0.1 to about 100 by weight, preferably in a ratio of from about 1 to about 50, and more preferably in a ratio of about 1 to about 10. Without being bound to any particular theory, the protecting agent reduces the adhesion between the therapeutic agent and the binder so that the therapeutic agent may be more easily released from the binder. In this way, the therapeutic agent may be delivered across the mucous membranes of the oral cavity within about 5 to about 20 minutes, preferably within about 10 minutes. Materials suitable as protecting agents are discussed in detail above and may be used alone or in combination in the tablet compositions of the present invention.

The tablet composition may also comprise one or more elastomeric solvents such as rosins and resins. Non-limiting examples of such solvents are discussed in detail above and may be used alone or in combination in the tablet compositions of the present invention. In addition, the tablet composition may further comprise waxes such as beeswax and microcrystalline wax, fats or oils such as soybean and cottonseed oil, and combinations thereof. Moreover, the tablet composition may additionally include plasticizers such as softeners or emulsifiers. Such plasticizers may, for example, help reduce the viscosity of the salivary solution of the dissolved tablet to a desirable consistency and improve its overall texture and bite and help facilitate the release of the therapeutic agent. Non-limiting examples of such plasticizers are discussed in detail above and may be used alone or in combination in the tablet compositions of the present invention.

In certain instances, the tablet composition includes a therapeutic agent centerfill. A centerfill may be particularly suitable when immediate release of the therapeutic agent is preferred. In addition, encapsulating the therapeutic agent in a centerfill may help to mask any undesirable taste that the therapeutic agent may have. In these instances, the binder surrounds, at least in part, a centerfill. The centerfill comprises at least one therapeutic agent, and may be a liquid or semi-liquid material. The centerfill material can be low-fat or fat free and contain one or more sweetening agents, flavoring agents, coloring agents, and/or scenting agents. Preferably, the centerfill includes a binary or ternary buffer system as described herein.

In certain other instances, the tablet composition of the present invention is multilayered. In this way, the dissolving or chewable tablet can be designed to provide more than one therapeutic agent, e.g., two or more 5-HT agonists or one or more 5-HT agonists in combination with one or more non-5-HT-agonist therapeutic agents. For example, with a bi-layered tablet, the first layer contains a 5-HT agonist and the second layer contains the same or different 5-HT agonist or a non-5-HT agonist therapeutic agent. Typically, the first layer comprises the dissolving or chewable portion of the tablet, and the second (i.e., subsequent) layer is coated by the first layer. This type of formulation may be particularly suitable when immediate release of the 5-HT agonist, followed by gastrointestinal absorption of a second therapeutic agent, is desirable. Gastrointestinal absorption of the second therapeutic agent may be desirable, for example, in order to mitigate co-morbid symptoms or to sustain the therapeutic benefit of the 5-HT agonist in the dissolving or the chewable portion of the tablet. Alternatively, the second layer is present as a layer lateral to the first layer. The second layer typically comprises at least one therapeutic agent, and can also comprise one or more sweetening agents, flavoring agents, coloring agents, and scenting agents as described above. In some instances, the second layer further includes a binary or ternary buffer system as described herein.

In still other instances, the combination of 5-HT agonists with or without non-5-HT agonist therapeutic agents need not take the form of a multilayered tablet, but instead comprises a single homogenous tablet layer. This type of formulation may also be used in the case where gastrointestinal absorption of at least one therapeutic agent is desirable. In this case, the relative extent of ionization of the two or more therapeutic agents determines how they are to be absorbed. For example, those therapeutic agents that are un-ionized are absorbed through the oral mucosa, while the ionized agents are swallowed for gastrointestinal absorption.

The tablet compositions can have any desired shape, size, and texture. For example, the tablet can have the shape of a stick, tab, pellet, sphere, and the like. Similarly, the tablet can be any desirable color. For example, the tablet can be any shade of red, blue, green, orange, yellow, violet, indigo, and mixtures thereof, and can be color coded to indicate the type and dosage of the therapeutic agent therein. The tablets can be individually wrapped or grouped together in pieces for packaging by methods well known in the art.

3. Lozenges

When the dosage form is a lozenge or candy, the compositions of the present invention comprise a 5-HT agonist or a pharmaceutically acceptable salt thereof, a carrier such as a binder, and a binary or ternary buffer system; The lozenge or candy composition may further comprise lubricating agents, wetting agents, emulsifying agents, suspending agents, preserving agents, sweetening agents, flavoring agents, coloring agents, and disintegrating agents. A general discussion of lozenges and candies is provided, e.g., in Pharmaceutical Dosage Forms, Volume 1: Tablets, 2^{nd} Ed., Marcel Dekker, Inc., New York, N.Y., pages 75-418 (1989).

Typically, the lozenge or candy compositions of the present invention comprise from about 0.001% to about 10.0% by weight of the 5-HT agonist (in whatever chosen form, measured as per its free base form), preferably from about 1.0% to about 5.0%, and more preferably from about 2.5% to about 4.5%. One skilled in the art understands that the foregoing percentages will vary depending upon the particular source of 5-HT agonist utilized, the amount of 5-HT agonist desired in the final formulation, as well as on the particular release rate of 5-HT agonist desired. The buffer system for the lozenge or candy composition is typically a binary or ternary buffer system comprising amorphous magnesium oxide with a carbonate salt and/or a bicarbonate salt. For example, a ternary buffer system typically comprises from about 4.0% to about 7.0% by weight sodium carbonate; from about 8.0% to about 12.0% by weight dessicant-coated sodium bicarbonate; and from about 20% to about 30% by weight amorphous magnesium oxide. The buffer system provides for a final salivary pH in excess of at least about 9.5, preferably at least about 9.9, and more preferably in the range of from about 9.9 to about 11. In a preferred embodiment, the lozenge or candy composition comprises about 3.5% by weight 5-HT agonist, about 5.5% by weight sodium carbonate, about 9.0% by weight dessicant-coated sodium bicarbonate, and about 25% by weight amorphous magnesium oxide. Examples of sumatriptan lozenge compositions are provided in Example 5 below.

In certain embodiments, the lozenge or candy is dissolved by a subject's saliva, without the need for chewing. For example, a lozenge placed on the subject's tongue can be used for buccal delivery of the therapeutic agent. Alternatively, a lozenge placed underneath the subject's tongue can be used for sublingual delivery of the therapeutic agent. This type of dosage form may be particularly desirable for pediatric and geriatric patients, since small children and aged individuals often have difficulty chewing certain items. Typically, the lozenge is formulated to dissolve within about 1 to about 15 minutes, preferably within about 2 to about 10 minutes, e.g., within about 2, 3, 4, 5, 6, 7, 8, 9, or 10 minutes, following administration. In a preferred embodiment, the lozenge or candy delivers the therapeutic agent across the sublingual mucosa.

As described above, the lozenges the present invention are typically formulated to dissolve within about 1 to 15 minutes following administration. However, while these time frames are amenable to maximum exposure of the therapeutic agent to the oral mucosa (e.g., to the sublingual and/or buccal mucosa), they are not always amenable to user compliance (e.g., users may swallow too, frequently and, therefore, hinder maximal transmucosal absorption). Consequently, in certain instances, it may be desirable to strike a balance between patient compliance and maximum exposure time of the therapeutic agent to the oral mucosa. This can be accomplished, for example, by reducing the lozenge size (e.g., from about 700-800 mg to about 200-300 mg) without reducing the concentration or the amount per unit dose of the buffer system or the therapeutic agent. In addition, subtle changes to the lozenge formulation such as, for example, replacing one flavoring agent for another (e.g., chocolate for spearmint) or replacing one binder or sweetening agent for another (e.g., lactose for mannitol or sorbitol) may be used to reduce salivation.

The carrier present in the lozenges of the present invention is typically a binder that is useful in keeping the lozenge in a semi-solid state, and may be a solid or a liquid, and may for example be a high-melting point fat or waxy material. Materials suitable as binders are discussed in detail above and may be used alone or in combination in the lozenge compositions of the present invention. In addition, binders such as mannitol, sorbitol, lactose, sucrose, and inositol can impart properties to the lozenge that permit or enhance its disintegration in the mouth.

The lozenge composition may further comprise a protecting agent. The protecting agent coats at least part of the therapeutic agent, typically upon the mixing of the two agents. The protecting agent may be mixed with the therapeutic agent in a ratio of from about 0.1 to about 100 by weight, preferably in a ratio of from about 1 to about 50, and more preferably in a ratio of about 1 to about 10. Without being bound to any particular theory, the protecting agent reduces the adhesion between the therapeutic agent and the binder so that the therapeutic agent may be more easily released from the binder. In this way, the therapeutic agent may be delivered across the mucous membranes of the oral cavity within about 5 to about 20 minutes, preferably within about 10 minutes. Materials suitable as protecting agents are discussed in detail above and may be used alone or in combination in the lozenge compositions of the present invention.

The lozenge composition may-also comprise one or more elastomeric solvents such as rosins and resins. Non-limiting examples of such solvents are discussed in detail above and may be used alone or in combination in the tablet compositions of the present invention. In addition, the lozenge composition may further comprise waxes such as beeswax and microcrystalline wax, fats or oils such as soybean and cottonseed oil, and combinations thereof. Moreover, the lozenge composition may additionally include plasticizers such as softeners or emulsifiers. Such plasticizers may, for example, help reduce the viscosity of the salivary solution of the dissolved lozenge to a desirable consistency and improve its overall texture and bite and help facilitate the release of the therapeutic agent. Non-limiting examples of such plasticizers are discussed in detail above and may be used alone or in combination in the lozenge compositions of the present invention.

In certain instances, the lozenge composition includes a therapeutic agent centerfill. A centerfill may be particularly suitable when immediate release of the therapeutic agent is preferred. In addition, encapsulating the therapeutic agent in a centerfill may help to mask any undesirable taste that the therapeutic agent may have. In these instances, the binder surrounds, at least in part, a centerfill. The centerfill comprises at least one therapeutic agent, and may be a liquid or semi-liquid material. The centerfill material can be a synthetic polymer, a semi-synthetic polymer, low-fat, or fat free and contain one or more sweetening agents, flavoring agents, coloring agents, and/or scenting agents. Preferably, the centerfill includes a binary or ternary buffer system as described herein.

In certain other instances, the lozenge composition of the present invention is multilayered. In this way, the lozenge can be designed to provide more than one therapeutic agent, e.g., two or more 5-HT agonists or one or more 5-HT agonists in combination with one or more non-5-HT agonist therapeutic agents. For example, with a bi-layered lozenge, the first layer contains a 5-HT agonist and the second layer contains the same or different 5-HT agonist or a non-5-HT agonist therapeutic agent. Typically, the first layer comprises the dissolving portion of the lozenge, and the second (i.e., subsequent) layer is coated by the first layer. This type of formulation may be particularly suitable when immediate release of the 5-HT agonist, followed by gastrointestinal absorption of a second therapeutic agent, is desirable. Gastrointestinal absorption of the second therapeutic agent may be desirable, for example, in order to mitigate co-morbid symptoms or to sustain the therapeutic benefit of the 5-HT agonist in the dissolving portion of the lozenge. Alternatively, the second layer is present as a layer lateral to the first layer. The second layer typically comprises at least one therapeutic agent, and can also comprise one or more sweetening agents, flavoring agents, coloring agents, and scenting agents as described above. In some instances, the second layer further includes a binary or ternary buffer system as described herein.

In still other instances, the combination of 5-HT agonists with or without non-5-HT agonist therapeutic agents need not take the form of a multilayered lozenge, but instead comprises a single homogenous lozenge layer. This type of formulation may also be used in the case where gastrointestinal absorption of at least one therapeutic agent is desirable. In this case, the relative extent of ionization of the two or more therapeutic agents determines how they are to be absorbed. For example, those therapeutic agents that are un-ionized are absorbed through the oral mucosa, while the ionized agents are swallowed for gastrointestinal absorption.

The lozenge compositions can have any desired shape, size, and texture. For example, the lozenge can have the shape of a stick, tab, pellet, sphere, and the like. Similarly, the lozenge can be any desirable color. For example, the lozenge can be any shade of red, blue, green, orange, yellow, violet, indigo, and mixtures thereof, and can be color coded to indicate the type and dosage of the therapeutic agent therein. The lozenges can be individually wrapped or grouped together in pieces for packaging by methods well known in the art.

In addition to the preferred dosage forms described above, the compositions of the present invention can also take to form of a solution formulation for delivery of a 5-HT agonist across the oral mucosa. For example, the solution formulation can be administered sublingually by using a two-chamber syringe delivery system, in which the upper chamber contains an unbuffered 5-HT agonist solution, the lower chamber contains the dry buffer system components, and a non-permeable membrane separates the upper and lower chambers. Depressing the syringe ruptures the non-permeable membrane and allows mixing of the unbuffered 5-HT agonist solution with the dry buffer system components. The resulting buffered 5-HT agonist solution is then released from the tip of the syringe. As such, by simply placing the tip of the syringe anywhere underneath a subject's tongue and depressing the syringe, a solution formulation of the present invention can be used to deliver the 5-HT agonist across the subject's sublingual mucosa.

Accordingly, the present invention further provides A composition for delivery of a 5-HT agonist across the oral mucosa, said composition comprising: (a) a 5-HT agonist or a pharmaceutically acceptable salt thereof, preferably sumatriptan; (b) a carrier; and (c) a binary buffer system comprising a carbonate salt and a bicarbonate salt, wherein the binary buffer system raises the pH of saliva to a pH greater than about 9.9 irrespective of the starting pH of saliva. Preferably, the composition is a solution that is prepared just prior to administration to the oral mucosa. In certain preferred embodiments, the binary buffer system comprises sodium bicarbonate and sodium carbonate wherein the ratio of sodium bicarbonate to sodium carbonate is from about 2:1 to about 5:1 by weight. In other embodiments, sodium carbonate is used in an amount that is equivalent to, or in excess of sodium bicarbonate. More particularly, the compositions are those that provide peak plasma levels of sumatriptan in less than 15 minutes (e.g, about 1-15 minutes), preferably in about 5 minutes to about 10 minutes.

D. Methods of Administration

The compositions of the present invention are useful in therapeutic applications, e.g., for treating a migraine. Importantly, the compositions of the present invention provide the rapid delivery of a 5-HT agonist across the oral mucosa by raising the pH of saliva to a pH greater than about 9.9, irrespective of the starting pH of saliva. In particular, the delivery of the therapeutic agent across the oral mucosa avoids hepatic first pass metabolism, degradation within the gastrointestinal tract, and drug loss during absorption. As a result, the therapeutic agent reaches the systemic circulation in a substantially shorter period of time and at a substantially higher concentration than with traditional oral (e.g., tablet) administration.

The compositions of the present invention have particular utility in the area of human and veterinary therapeutics. Generally, administered dosages will be effective to deliver picomolar to micromolar concentrations of the 5-HT agonist to the appropriate site.

Administration of the compositions of the present invention is preferably carried out via any of the accepted modes of administration to the mucous membranes of the oral cavity. Examples of suitable sites of administration within the oral mucosa include, without limitation, the mucous membranes of the floor of the mouth (sublingual mucosa), the cheeks (buccal mucosa), the gums (gingival mucosa), the roof of the mouth (palatal mucosa), the lining of the lips, and combinations thereof. These regions differ from each other with respect to their anatomy, drug permeability, and physiological response to drugs. Preferably, the compositions of the present invention are administered to the sublingual mucosa, buccal mucosa, or a combination thereof.

The oral mucosa, possessing a rich blood supply and suitable drug permeability, is an especially attractive route of administration for systemic drug delivery. Furthermore, delivery of a therapeutic agent across the oral mucosa bypasses hepatic first pass metabolism, avoids enzymatic degradation within the gastrointestinal tract, and provides a more suitable enzymatic flora for drug absorption. As used herein, the term “sublingual delivery” refers to the administration of a therapeutic agent across the mucous membranes lining the floor of the mouth and/or the ventral tongue. The term “buccal delivery” as used herein refers to the administration of a therapeutic agent across the mucous membranes lining the cheeks.

The oral mucosa is composed of an outermost layer of stratified squamous epithelium. Beneath this layer lies a basement membrane, i.e., the lamina propria, followed by the submucosa as the innermost layer. The epithelium of the oral mucosa is similar to the stratified squamous epithelia found in the rest of the body in that it contains a mitotically active basal cell layer, advancing through a number of differentiating intermediate layers to the superficial layers, where cells are shed from the surface of the epithelium (Gandhi et al., Ind. J. Pharm. Sci., 50:145-152 (1988)). For example, the epithelium of the buccal mucosa is about 40-50 cell layers thick, while that of the sublingual epithelium contains somewhat fewer cell layers. The epithelial cells increase in size and become flatter as they travel from the basal layers to the superficial layers.

The turnover time for buccal mucosal epithelium, estimated at 5-6 days, is representative of the turnover time for sublingual mucosal epithelium as well as other epithelia in the oral mucosa (Harris et al., J. Pharm. Sci, 81:1-10 (1992)). The thickness of the oral mucosa varies depending on the site in the oral cavity. For example, the buccal mucosa measures at about 500-800 μm in thickness, while the hard and soft palatal mucosa, the sublingual mucosa, the ventral tongue, and the gingival mucosa measure at about 100-200 μm in thickness. The composition of the epithelium also varies depending on the site in the oral cavity. For example, the mucosae of areas subject to mechanical stress (i.e., the gingivae and hard palate) are keratinized similar to the epidermis. However, the mucosae of the soft palate, the sublingual region, and the buccal region are not keratinized (Harris et al., supra). The keratinized epithelia contain neutral lipids like ceramides and acylceramides, which have been associated with providing a barrier function. As a result, these epithelia are relatively impermeable to water. In contrast, non-keratinized epithelia, such as sublingual and buccal epithelia, do not contain acylceramides and have only small amounts of ceramide (Wertz et al., Crit. Rev. Ther. Drug Carr. Sys., 8:237-269 (1991); Squier et al., J. Invest. Dermat., 96:123-126 (1991); Squier et al., in Oral Mucosal Drug Delivery, Ed. M. J. Rathbone, Marcel Dekker, Inc., New York, N.Y., 1-26 (1996)). Non-keratinized epithelia also contain small amounts of neutral but polar lipids, e.g., cholesterol sulfate and glucosyl ceramides. As such, these epithelia have been found to be considerably more permeable to water than keratinized epithelia (Harris et al., supra; Wertz et al., supra; Squier et al, supra, 1991).

In general, the oral mucosa is a somewhat leaky epithelia intermediate between that of the epidermis and intestinal mucosa. For example, the permeability of the buccal mucosa is estimated to be about 4-4000 times greater than that of skin (Galey et al., J. Invest. Dermat., 67:713-717 (1976)). The permeability of different regions of the oral mucosa generally decrease in the order of sublingual mucosa greater than buccal mucosa, and buccal mucosa greater than palatal mucosa (Harris et al., supra). This permeability is generally based upon the relative thickness and degree of keratinization of these membranes, with the sublingual mucosa being relatively thin and non-keratinized, the buccal mucosa being thicker and non-keratinized, and the palatal mucosa being intermediate in thickness, but keratinized.

The epithelial cells of the oral mucosa are surrounded by mucus comprising primarily complexes of proteins and carbohydrates that may or may not be attached to certain regions on the cell surface. The mucus may play a role in cell-cell adhesion, as well as acting as a lubricant, allowing cells to move relative to one another (Tabak et al., J. Oral Pathol., 11:1-17 (1982)). In stratified squamous epithelia found elsewhere in the body, mucus is synthesized by specialized mucus secreting cells such as goblet cells; however, in the oral mucosa, mucus is secreted by the major and minor salivary glands as part of saliva (Tabak et al., supra; Rathbone et al., Adv. Drug Del. Rev., 13:1-22 (1994)). At physiological pH, the mucus network carries a negative charge due to the sialic acid and sulfate residues present on the carbohydrates. At this pH, mucus can form a strongly cohesive gel structure that binds to the epithelial cell surface as a gelatinous layer (Gandhi et al., supra). Without being bound to any particular theory, the buffer systems of the present invention neutralize the sialic acid residues present on the carbohydrates and prevent them from interacting with the therapeutic agent, thereby further enhancing drug permeation.

Another feature of the environment of the oral cavity is the presence of saliva produced by the salivary glands. Saliva is the protective fluid for all tissues of the oral cavity. Saliva is an aqueous fluid with about 1% organic and inorganic materials. The major determinant of the salivary composition is the flow rate, which in turn depends upon factors such as the time of day, the type of stimulus, and the degree of stimulation. The salivary pH typically ranges from about 5.5 to about 7.0, depending on the flow rate. For example, at high flow rates, the sodium and bicarbonate concentrations increase, leading to an increase in the pH. Because the daily salivary volume is between about 0.5 to about 2 liters, the oral cavity provides an aqueous environment for the hydration and/or dissolution of the oral mucosal dosage forms of the present invention.

The sublingual mucosa is the most highly permeable region of the oral cavity, and provides rapid absorption and high bioavailability of a drug in a convenient, accessible, and well-accepted route of administration (Harris et al., supra). Suitable sublingual dosage forms include, without limitation, tablets (e.g., quick-dissolving, slow-dissolving), lozenges, candy, and soft gelatin capsules filled with liquid drug. Such systems create a very high drug concentration in the sublingual region before they are systemically absorbed across the sublingual mucosa. As a result, the sublingual mucosa is particularly well-suited for producing a rapid onset of action, and sublingual dosage forms can be used to deliver drugs with shorter delivery period requirements and/or less frequent dosing regimens. Although the buccal mucosa is considerably less permeable than the sublingual area, rapid absorption and high bioavailability of a drug can also be observed with buccal administration. Suitable buccal dosage forms include, without limitation, chewing gums, tablets (e.g., quick-dissolving, slow-dissolving), lozenges, candy, and the like. Both the buccal mucosa and the sublingual mucosa are far superior to the gastrointestinal tract for providing increased absorption and bioavailability of a drug.

To increase the permeability of drugs through the oral mucosa, penetration enhancers can be included in the dosage forms of the present invention. The penetration enhancers may be of the type that alters the nature of the oral mucosa to enhance penetration, or of the type that alters the nature of the therapeutic agent to enhance penetration through the oral mucosa. Suitable penetration enhancers include, without limitation, polyoxyethylene 23-lauryl ether, aprotin, azone, benzalkonium chloride, cetylpyridinium chloride, cetyltrimethylammonium bromide, cyclodextrin, dextran sulfate, lauric acid, propylene glycol, lysophosphatidylcholine, menthol, methoxysalicylate, methyloleate, oleic acid; phosphatidylcholine, polyoxyethylene, polysorbate 80, sodium ethylenediaminetetraacetic acid (“EDTA”), sodium deoxycholate, sodium glycocholate, sodium glycodeoxycholate, sodium lauryl suflate, sodium salicylate, sodium taurocholate, sodium taurodeoxycholate, as well as certain sulfoxides and glycosides, and combinations thereof.

### IV. EXAMPLES

The following examples are offered to illustrate, but not to limit, the claimed invention.

### Example 1

### Sumatriptan Membrane Assay

This example illustrates the beneficial effects of pH adjustment on membrane penetration for a sumatriptan dosage form.

The effect of pH adjustment on the extent of ionization, and hence, the extent to which a therapeutic agent will traverse the mucous membrane can be demonstrated using a membrane assay; see, e.g., Kansy et al., J. Med. Chem., 41:1007-1010(1998); and Avdeef, Curr. Topics Med. Chem., 1:277-351 (2001). This assay uses a lipid-coated membrane to predict lipid mucosal membrane penetration. The membrane apparatus consists of a dodecane membrane sandwiched between a donor and acceptor cell. The lipid-coated membrane is less porous then the mucous membrane of the oral cavity. Thus, the enhancement seen in the membrane assay is very likely to be magnified in vivo.

The dissociation constant (pKa) of sumatriptan is 9.5, and therefore the drug would be 100% un-ionized at pH 11.5 and 90% at pH 10.5. Membrane assays were performed using sumatriptan succinate at pH values of 9.0, 9.5, and 10.0. The final pH values of these solutions were adjusted using freshly prepared 0.01 M sodium bicarbonate/sodium carbonate buffer. Permeation was measured by determining the concentration of sumatriptan in the acceptor cell, and is expressed as Pₑ (effective permeability in centimeters per second). As shown in Table 3 below, the effective permeability of sumatriptan increased with pH.

TABLE 3
Effective permeability (Pₑ) of sumatriptan in a membrane assay.

| pH | Pₑ (cm/s) |
|---|---|
| 9.0 | 5.99 |
| 9.5 | 9.45 |
| 10.0 | 15.61 |

### Example 2

### Sumatriptan Pharmacokinetic Study

This example illustrates the pharmacokinetic profile of a sumatriptan solution of the present invention as compared to a dose equivalent commercial oral tablet.

Because the lipid-coated membrane is less porous than the mucous membrane of the oral cavity, the enhancement seen in the membrane assay is very likely to be magnified in situ, resulting in enhanced buccal absorption and higher bioavailability of sumatriptan relative to a dose equivalent commercial oral tablet. To evaluate the pharmacokinetic profile of a buccally administered sumatriptan formulation, a 25 mg sumatriptan succinate solution buffered at pH 10 with 150 mg sodium bicarbonate and 50 mg sodium carbonate (Formulation A) was compared to a dose equivalent commercial oral tablet formulation (Formulation B), i.e., Imitrex® (GlaxoSmithKline; Research Triangle Park, N.C.), in four healthy subjects following a 10 hour overnight fast. Subject demographics are shown in Table 4 below.

TABLE 4
Subject demographics.

| | |
|---|---|
| Number of Subjects | 4 (1 female; 3 male) |
| Average Age (yr) | 32 (min. 19; max. 45) |
| Frame | medium |
| Average Weight (kg) | 75 (min. 62; max. 82) |
| Average Height (cm) | 176 (min. 165; max. 182) |

A single dose, open label, randomized, two treatment; two-way crossover study with a three day washout period between treatments was performed. The sample size used in this study is typical for assessing safety/tolerability (Simon et al., J. Natl. Cancer Inst., 89:1138-1147 (1997)) and for comparing routes of administration (Chang et al., Ann. Pharmacother., 33:781-786 (1999)). Furthermore, due to the crossover nature of this study, each subject acts as his or her own control. As a result, variables such as age, gender, and differences in physiology and enzymology are controlled; see, e.g., Chow et al., Marcel Dekker, 1992, page 30). For Formulation A, blood samples were taken at 0, 5, 10, 15, 20, 30, 45, 60, 90, 120, 180, 240, and 360 minutes following administration. For Formulation B, blood samples were taken at 0, 10, 20, 30, 45, 60, 90, 120, 180, 240, and 360 minutes following administration. High pressure liquid chromatography (HPLC)-tandem mass spectrometry (MS) assays were performed to determine plasma sumatriptan levels. The assay parameters are shown in Table 5 below.

TABLE 5
Sumatriptan assay parameters.

| | |
|---|---|
| Molecular Ions Analyzed | 296.4 (parent); 58 (daughter) |
| Calibration Curve Concentration | 1-200 ng/ml plasma |
| Range | |
| Curve and Coefficient of | Power curve (log—log straight |
| Correlation | line) with linearity r² > 0.996 |
| Minimum Detectable Concentration | 0.1 ng/ml |
| Typical Plasma Volume | 100 μl |

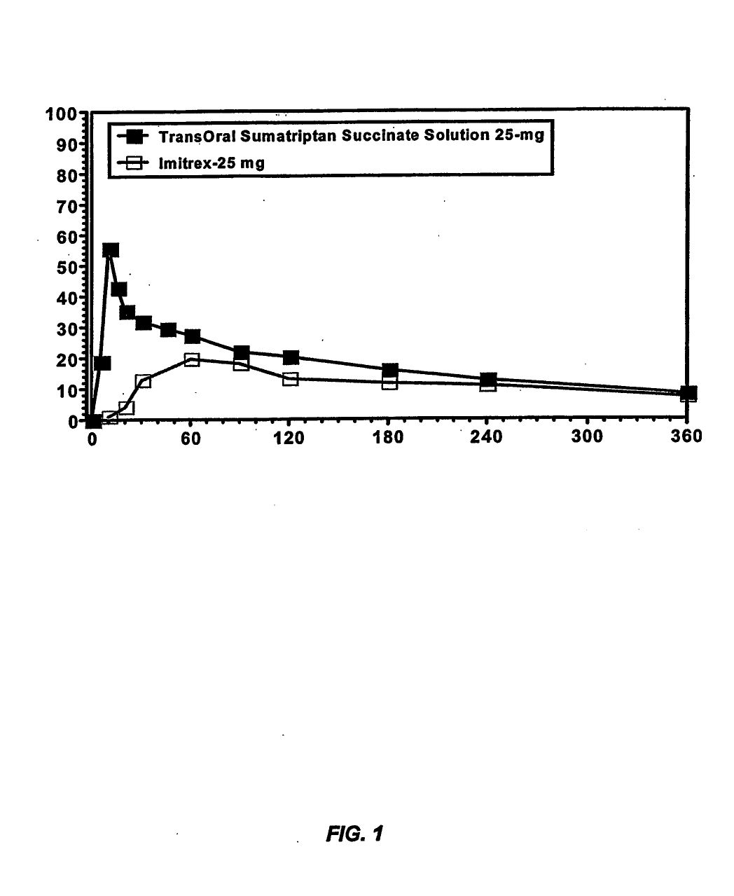

FIG. 1 shows the mean plasma concentration over time for Formulation A (25 mg buccal sumatriptan succinate solution) and Formulation B (25 mg sumatriptan succinate oral tablet). Table 6 below shows the pharmacokinetic parameters determined for both formulations. This study demonstrates that delivery of sumatriptan across the oral mucosa produced plasma sumatriptan concentrations that were over three times greater than those observed for the commercial oral tablet during the hour immediately following administration. In addition, peak plasma sumatriptan concentrations were achieved within 10 minutes following buccal administration, while peak plasma sumatriptan concentrations were not achieved until 60 minutes following commercial oral tablet administration. As such, the present study shows that sumatriptan from the buffered solution is rapidly absorbed and has substantially better bioavailability than the commercial oral tablet.

TABLE 6
Pharmacokinetic parameters for Formulation
A and Formulation B.

| | Formulation A | |
|---|---|---|
| | (sumatriptan succinate | Formulation B |
| Formulation | solution) | (Imitrex ®) |
| Cₘₐₓ (ng/ml) | 55.6 ± 24.2 | 20.1 ± 5.2 |
| | (31.3; 88.3) | (15.2; 27.5) |
| Tₘₐₓ (hr) | 0.17 | 1.06 ± 0.31 |
| | | (0.8; 1) |
| AUC_{0-6 hr} (ng · hr/ml) | 107.4 ± 37.8 | 68.9 ± 12.1 |
| | (79.8; 160.9) | (57.7; 84.5) |
| AUC_{0-1 hr} (ng · hr/ml) | 31.9 ± 11.5 | 10.2 ± 2.1 |
| | (21.6; 47.9) | (8.1; 13.2) |

Values represent the mean ± standard deviation (SD). The numbers in parentheses represent the minimum and maximum values, respectively.

### Example 3

### Sumatriptan Gum Compositions

This example illustrates the sumatriptan chewing gum compositions of the present invention.

Sumatriptan can be formulated as a chewing gum composition as described above. In these embodiments, the unit dose or serving of the chewing gum comprises from about 0.1 to about 100 milligrams (mg) sumatriptan (as measured in its free base form), preferably from about 1 to about 50 mg, and more preferably from about 2 to about 25 mg. In other embodiments, the unit dose comprises from about 2 to about 20 mg sumatriptan, preferably from about 5 to about 15 mg. Extra sumatriptan, for example, up to from about 10% to about 25% by weight, can be added as “overage” or as the amount that may be expected to be “washed away” and not otherwise released or absorbed during mastication.

Given in weight percentages, the sumatriptan chewing gum composition comprises from about 0.001% to about 2.0% sumatriptan (in whatever chosen form, measured as per its free base form), and preferably from about 0.002% to about 1.0%. In some embodiments, about 0.008% sumatriptan is used. One skilled in the art understands that the foregoing percentages will vary depending upon the particular source of sumatriptan utilized, the amount of sumatriptan desired in the final formulation, as well as on the particular release rate of sumatriptan desired. The buffer system of the sumatriptan chewing gum composition provides for a final salivary pH in excess of at least about 9.5, preferably at least about 9.9, and more preferably in the range of from about 9.9 to about 11.

A sumatriptan chewing gum was made according to the following procedure. Silicon dioxide USP (0.35 kg) was passed through a #20 mesh screen and then loaded into a blender containing 0.810 kg mannitol granular USP and 9.430 kg Pharmagum C. The material was blended for 10 minutes. Sumatriptan succintate EP (0.173 kg) was ground with silicon dioxide (0.02 kg) using a mortar and pestle. The remaining silicon dioxide, along with 0.228 kg magnesium stearate, was added into the mortar while continuing to grind. The ground materials were transferred into a plastic bag, and the mortar was rinsed using 0.01 kg silicone dioxide, and transferred into the bag. The contents of the bag were then blended for five minutes.

Equal parts of the blended bag contents and the blended mannitol gum base mixture were blended for an additional five minutes. This process was repeated until all the sumatriptan and gum base mixture had been blended together. Sodium carbonate (0.110 kg), sodium bicarbonate (0.570 kg), gum acacia (0.43 kg), xanthan gum (0.013 kg), and aspartame (0.072 kg), were then loaded into the blender with natural and artificial flavors and blended for ten minutes with 0.090 kg of silicon dioxide. The flavors used were as follows: natural and artificial grape flavor S.D. (0.215 kg); natural and articial cherry flavor (0.108 kg); natural and artificial fruit punch flavor S.D. (0.180 kg); natural cherry WONF DURAROME® flavor (0.215 kg); and natural passion fruit type DURAROME® flavor (0.035 kg).

The blend was passed through a #12 mesh screen and then blended for an additional 15 minutes. Magnesium stearate (0.114 kg) was passed through a #20 mesh screen, added to the blend, and blended for five minutes. The blend was collected and placed in plastic bags. Two silica gel desiccant bags were placed around the plastic bags to absorb ambient moisture. The blend was then compressed and compacted using a tablet press. By using the above described procedure, the average particle size of the drug (i.e., sumatriptan) in the chewing gum is about 20 microns, as compared to a typical average drug particle size of from about 75 to about 100 microns. In addition, the average particle size of the drug in the chewing gum is less than or equal to the average particle size of the carrier ingredients (e.g., gum base, binders, etc.).

The sumatriptan chewing gum compositions of the present invention can be used, e.g., for treating a migraine. After introduction of a serving size piece of the gum composition into the mouth, the subject chews the gum as is normally done with any non-medicated type of chewing gum for about 20-30 minutes, at approximately an average rate of about 10-45 chews per minute. The gum is then discarded.

A serving of the sumatriptan chewing gum is typically designed to cause a loaded sumatriptan concentration level in the bloodstream of at least about 5 to about 300 nanograms (ng) of sumatriptan per milliliter (ml) of plasma. The ratio of the maximum plasma concentration (Cₘₐₓ) to the time to achieve that maximum plasma concentration (Tₘₐₓ) is preferably within a range of about 10 ng/ml×hr to about 1000 ng/ml×hr, and more preferably within a range of about 100 ng/ml×hr to about 500 ng/ml×hr. The chewing gum compositions of the present invention provide a convenient, reliable, practical, and painless system for delivering sumatriptan across the oral mucosa. Notably, the chewing gum compositions are capable of rapidly delivering sumatriptan so that a therapeutically effective amount of sumatriptan enters the bloodstream within 20 minutes, 10 minutes, or even within 1-2 minutes after sumatriptan is released from the carrier.

### Example 4

### Sumatriptan Quick-Dissolving Tablet Compositions

This example illustrates the quick-dissolving sumatriptan tablet compositions of the present invention.

Given in weight percentages, the sumatriptan quick-dissolving tablet composition typically comprises from about 0.001% to about 10.0% sumatriptan (in whatever chosen form, measured as per its free base form), and more typically from about 1.0% to about 5.0%. In some embodiments, about 3.5% sumatriptan is used. One skilled in the art understands that the foregoing percentages will vary depending upon the particular source of sumatriptan utilized, the amount of sumatriptan desired in the final formulation, as well as on the particular release rate of sumatriptan desired. The buffer system of the sumatriptan quick-dissolving tablet composition provides for a final salivary pH in excess of at least about 9.5, preferably at least about 9.9, and more preferably in the range of from about 9.9 to about 11.

A sumatriptan quick-dissolving tablet was made according to the following procedure. Mannitol (3.633 kg) and sorbitol (0.330 kg) were blended for ten minutes. Sodium bicarbonate (0.330 kg), sodium carbonate (0.165 kg), natural peppermint flavor (0.125 kg), natural menthol flavor (0.025 kg), and sucralose (0.020 kg) were blended separately for ten minutes. Stearic acid (0.125 kg), magnesium stearate (0.075 kg), and sumatriptan succinate (0.172 kg) were blended for ten minutes and then passed through a #12 mesh screen. The blended mixtures were then added together and compressed into tablets. By using this procedure, the average particle size of the drug (i.e., sumatriptan) in the quick-dissolving tablet is about 20 microns, as compared to a typical average drug particle size of from about 75 to about 100 microns. In addition, the average particle size of the drug in the quick-dissolving tablet is less than or equal to the average particle size of the carrier ingredients (e.g., gum base, binders, etc.).

The sumatriptan quick-dissolving tablets of the present invention can be used, e.g., for treating a migraine. As such, the quick-dissolving tablets provide a convenient, reliable, practical, and painless system for delivering sumatriptan across the oral mucosa. Notably, the quick-dissolving tablets are capable of rapidly delivering sumatriptan so that a therapeutically effective amount of sumatriptan enters the bloodstream within 20 minutes, 10 minutes, or even within 1-2 minutes after sumatriptan is released from the carrier.

### Example 5

### Sumatriptan Lozenge Compositions

This example illustrates the sumatriptan lozenge compositions of the present invention.

Given in weight percentages, the sumatriptan lozenge composition typically comprises from about 0.001% to about 10.0% sumatriptan (in whatever chosen form, measured as per its free base form), preferably from about 1.0% to about 5.0% sumatriptan, and more preferably from about 2.5% to about 4.5% sumatriptan. The buffer system for the sumatriptan lozenge composition is typically a binary or ternary buffer system comprising amorphous magnesium oxide with a carbonate salt and/or a bicarbonate salt. For example, a ternary buffer system typically comprises from about 4.0% to about 7.0% sodium carbonate; from about 8.0% to about 12.0% dessicant-coated sodium bicarbonate; and from about 20% to about 30% amorphous magnesium oxide. In a preferred embodiment, the sumatriptan lozenge composition comprises about 3.5% sumatriptan. about 5.5% sodium carbonate, about 9.0% dessicant-coated sodium bicarbonate, and about 25% amorphous magnesium oxide. The buffer system provides for a final salivary pH in excess of at least about 9.5, preferably at least about 9.9, and more preferably in the range of from about 9.9 to about 11.

A sumatriptan sublingual lozenge was made according to the formulation shown in Table 7. Briefly, mannitol and sorbitol were blended. Sodium carbonate, dessicant-coated sodium bicarbonate, magnesium oxide, natural and artificial spearmint flavor, and sucralose were blended separately. Magnesium stearate and sumatriptan were blended and then passed through a #12 mesh screen. The blended mixtures were then added together and compressed to produce white round lozenges. By using this procedure, the average particle size of the drug (i.e., sumatriptan) in the lozenge is about 20 microns, as compared to a typical average drug particle size of from about 75 to about 100 microns. In addition, the average particle size of the drug in the lozenge is less than or equal to the average particle size of the carrier ingredients (e.g., gum base, binders, etc.). The unit weight for each lozenge was 250 mg.

TABLE 7
Sumatriptan lozenge formulation.

| | Unit | Batch |
|---|---|---|
| | Quantity | Quantity |
| Material | (mg) | (g) |
| Sodium Carbonate, NF | 14.000 | 294.000 |
| Sodium Bicarbonate (Effer Soda) | 23.000 | 483.000 |
| Sumatriptan | 9.000 | 189.000 |
| Mannogem EZ (Mannitol), USP | 40.000 | 840.000 |
| Sorbogem 712 (Sorbitol), NF | 80.000 | 1680.000 |
| Magnesium Oxide @ Mg = 57% | 63.400 | 1331.400 |
| Natural & Artificial Spearmint Flavor | 6.500 | 136.500 |
| Sucralose, NF | 1.100 | 23.100 |
| Silicon Dioxide, USP | 5.500 | 115.500 |
| Magnesium Stearate, NF | 7.500 | 157.500 |

The batch quantity formulation produces 21,000 unit doses.

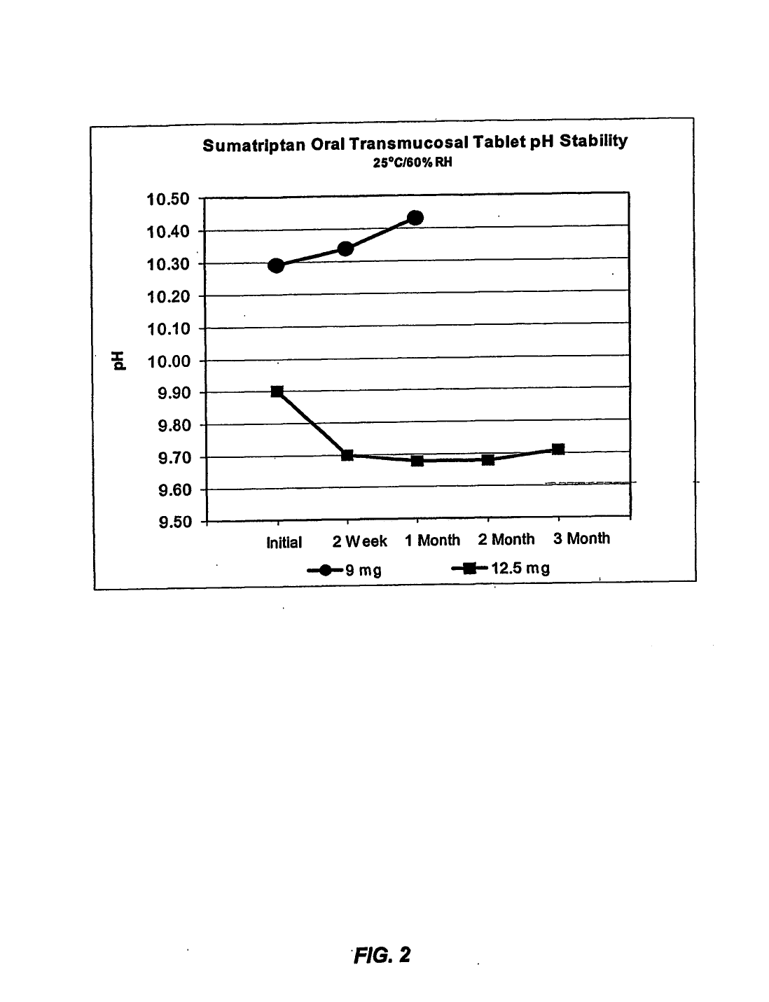

As shown in FIG. 2, the sublingual lozenge containing 9 mg sumatriptan had a pH of above 10.2 and maintained a stable pH at 25° C and 60% relative humidity (RH). A sublingual lozenge containing 12.5 mg sumatriptan had a pH of about 9.9 and maintained a stable pH (see, FIG. 2).

The sumatriptan lozenges of the present invention can be used, e.g., for treating a migraine. As such, the lozenges provide a convenient, reliable, practical, and painless system for delivering sumatriptan across the oral mucosa. For example, the sumatriptan lozenges are simply kept in the mouth (i.e., under the tongue) for about two or more minutes. Preferably, the sumatriptan lozenges dissolve within about 6 minutes following administration.

The use of a ternary buffer system comprising sodium carbonate, sodium bicarbonate, and magnesium oxide in the lozenges of the present invention raises the pH of saliva to a pH greater than about 9.9 irrespective of the starting pH of saliva, thus allowing for the rapid delivery across the oral mucosa (e.g., sublingual mucosa) of a therapeutically effective amount of sumatriptan. As a result, sumatriptan enters the bloodstream within 20 minutes, 10 minutes, or even within 1-2 minutes after being released from the carrier. Notably, the magnesium oxide in the ternary buffer system serves several important functions including, for example, raising the pH of the formulation, masking the corrosiveness of sodium carbonate, serving as a secondary binding agent thereby eliminating the need for stearic acid, and lowering the amount of sodium carbonate needed to produce the desired pH.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A composition for delivery of a 5-HT agonist across the oral mucosa, said composition comprising:
(a) a 5-HT agonist or a pharmaceutically acceptable salt thereof;
(b) a carrier; and
(c) a ternary buffer system comprising a carbonate salt, a bicarbonate salt, and a metal oxide,
wherein said ternary buffer system raises the pH of saliva to a pH greater than about 9.9 irrespective of the starting pH of saliva.

2. A composition of claim 1, wherein said ternary buffer system raises the pH of saliva to a pH of from about 9.9 to about 11 irrespective of the starting pH of saliva.

3. A composition of claim 1, wherein said 5-HT agonist is selected from the group consisting of sumatriptan, naratriptan, rizatriptan, eletriptan, almotriptan, zolmitriptan, frovatriptan, and combinations thereof.

4. A composition of claim 1, wherein said carbonate salt is selected from the group consisting of sodium carbonate and potassium carbonate.

5. A composition of claim 1, wherein said bicarbonate salt is selected from the group consisting of sodium bicarbonate and potassium bicarbonate.

6. A composition of claim 1, wherein said metal oxide is selected from the group consisting of magnesium oxide and aluminum oxide.

7. A composition of claim 6, wherein said magnesium oxide is amorphous magnesium oxide.

8. A composition of claim 1, wherein said ternary buffer system comprises sodium carbonate, sodium bicarbonate, and amorphous magnesium oxide.

9. A composition of claim 1, wherein said carrier is selected from the group consisting of a binder, a gum base, and combinations thereof.

10. A composition of claim 9, wherein said gum base comprises at least one hydrophobic polymer and at least one hydrophilic polymer.

11. A composition of claim 9, wherein said binder is selected from the group consisting of a sugar, a sugar alcohol, and combinations thereof.

12. A composition of claim 11, wherein said sugar alcohol is selected from the group consisting of mannitol, sorbitol, xylitol, and combinations thereof.

13. A composition of claim 1, wherein said composition is a dosage form selected from the group consisting of a lozenge, a chewing gum, a chewable tablet, and a dissolving tablet.

14. A composition of claim 13, wherein said dissolving tablet is selected from the group consisting of a slow-dissolving tablet and a quick-dissolving tablet.

15. A composition of claim 1, wherein said oral mucosa is selected from the group consisting of the sublingual mucosa, the buccal mucosa, and a combination thereof.

16. A composition of claim 1, further comprising a 5-HT antagonist.

17. A composition of claim 1, further comprising a non-steroidal anti-inflammatory drug (NSAID).

18. A composition of claim 1, wherein the average particle size of said 5-HT agonist or a pharmaceutically acceptable salt thereof is less than or equal to the average particle size of said carrier.

19. A composition of claim 1, wherein said 5-HT agonist is sumatriptan and said ternary buffer system comprises sodium carbonate, sodium bicarbonate, and amorphous magnesium oxide.

20. A composition of claim 19, wherein said composition is a lozenge or a dissolving tablet.

21. A composition of claim 20, wherein said composition is administered sublingually.

22. A composition of claim 19, wherein said sodium bicarbonate is dessicant-coated sodium bicarbonate.

23. A composition of claim 19, wherein the weight percent of amorphous magnesium oxide is greater than the combined weight percent of sodium carbonate and sodium bicarbonate.

24. A composition of claim 23, wherein said composition comprises from about 2.5 to about 4.5 weight percent sumatriptan; from about 4.0 to about 7.0 weight percent sodium carbonate; from about 8.0 to about 12.0 weight percent dessicant-coated sodium bicarbonate; and from about 20 to about 30 weight percent amorphous magnesium oxide.

25. A composition of claim 24, wherein composition comprises about 3.5 weight percent sumatriptan; about 5.5 weight percent sodium carbonate; about 9.0 weight percent dessicant-coated sodium bicarbonate; and about 25 weight percent amorphous magnesium oxide.

26. A composition for delivery of a 5-HT agonist across the oral mucosa, said composition comprising:
(a) a 5-HT agonist or a pharmaceutically acceptable salt thereof;
(b) a carrier; and
(c) a ternary buffer system comprising a carbonate salt, a bicarbonate salt, and a citrate, phosphate, or borate salt,
wherein said ternary buffer system raises the pH of saliva to a pH greater than about 9.9 irrespective of the starting pH of saliva.

27. A composition of claim 26, wherein said ternary buffer system raises the pH of saliva to a pH of from about 9.9 to about 11 irrespective of the starting pH of saliva.

28. A composition of claim 26, wherein said 5-HT agonist is selected from the group consisting of sumatriptan, naratriptan, rizatriptan, eletriptan, almotriptan, zolmitriptan, frovatriptan, and combinations thereof.

29. A composition of claim 26, wherein said carbonate salt is selected from the group consisting of sodium carbonate and potassium carbonate.

30. A composition of claim 26, wherein said bicarbonate salt is selected from the group consisting of sodium bicarbonate and potassium bicarbonate.

31. A composition of claim 26, wherein said citrate salt is selected from the group consisting of sodium citrate, potassium citrate, calcium citrate, magnesium citrate, and ammonium citrate.

32. A composition of claim 26, wherein said phosphate salt is selected from the group consisting of monobasic sodium phosphate, dibasic sodium phosphate, monobasic potassium phosphate, dibasic potassium phosphate, monobasic calcium phosphate, dibasic calcium phosphate, monobasic magnesium phosphate, dibasic magnesium phosphate, monobasic ammonium phosphate, and dibasic ammonium phosphate.

33. A composition of claim 26, wherein said borate salt is selected from the group consisting of sodium borate, potassium borate, calcium borate, magnesium borate, and ammonium borate.

34. A composition of claim 26, further comprising a metal oxide.

35. A composition of claim 26, wherein said carrier is selected from the group consisting of a binder, a gum base, and combinations thereof.

36. A composition of claim 26, wherein said composition is a dosage form selected from the group consisting of a lozenge, a chewing, gum, a chewable tablet, and a dissolving tablet.

37. A composition of claim 36, wherein said dissolving tablet is selected from the group consisting of a slow-dissolving tablet and a quick-dissolving tablet.

38. A composition of claim 26, wherein said oral mucosa is selected from the group consisting of the sublingual mucosa, the buccal mucosa, and a combination thereof.

39. A composition of claim 26, wherein the average particle size of said 5-HT agonist or a pharmaceutically acceptable salt thereof is less than or equal to the average particle size of said carrier.

40. A composition of claim 26, wherein said 5-HT agonist is sumatriptan and said ternary buffer system comprises sodium carbonate, sodium bicarbonate, and a citrate, phosphate, or borate salt.

41. A composition of claim 40, wherein said composition is a lozenge or a dissolving tablet.

42. A composition of claim 41, wherein said composition is administered sublingually.

43. A composition for delivery of a 5-HT agonist across the oral mucosa, said composition comprising:
(a) a 5-HT agonist or a pharmaceutically acceptable salt thereof;
(b) a carrier; and
(c) a buffer system comprising a carbonate salt or a bicarbonate salt and two or more buffering agents selected from the group consisting of a metal oxide, a citrate salt, a phosphate salt, and a borate salt,
wherein said buffer system raises the pH of saliva to a pH greater than about 9.9 irrespective of the starting pH of saliva.

44. A composition of claim 43, wherein said ternary buffer system raises the pH of saliva to a pH of from about 9.9 to about 11 irrespective of the starting pH of saliva.

45. A composition of claim 43, wherein said 5-HT agonist is selected from the group consisting of sumatriptan, naratriptan, rizatriptan, eletriptan, almotriptan, zolmitriptan, frovatriptan, and combinations thereof.

46. A composition of claim 43, wherein said carbonate salt is selected from the group consisting of sodium carbonate and potassium carbonate.

47. A composition of claim 43, wherein said bicarbonate salt is selected from the group consisting of sodium bicarbonate and potassium bicarbonate.

48. A composition of claim 43, wherein said carrier is selected from the group consisting of a binder, a gum base, and combinations thereof.

49. A composition of claim 43, wherein said composition is a dosage form selected from the group consisting of a lozenge, a chewing gum, a chewable tablet, and a dissolving tablet.

50. A composition of claim 49, wherein said dissolving tablet is selected from the group consisting of a slow-dissolving tablet and a quick-dissolving tablet.

51. A composition of claim 43, wherein said oral mucosa is selected from the group consisting of the sublingual mucosa, the buccal mucosa, and a combination thereof.

52. A composition of claim 43, wherein the average particle size of said 5-HT agonist or a pharmaceutically acceptable salt thereof is less than or equal to the average particle size of said carrier.

53. A composition of claim 43, wherein said composition is administered sublingually.

54. A composition for delivery of a 5-HT agonist across the oral mucosa, said composition comprising:
(a) a 5-HT agonist or a pharmaceutically acceptable salt thereof;
(b) a carrier; and
(c) a binary buffer system comprising a carbonate salt or a bicarbonate salt and a metal oxide,
wherein said binary buffer system raises the pH of saliva to a pH greater than about 9.9 irrespective of the starting pH of saliva.

55. A composition of claim 54, wherein said binary buffer system raises the pH of saliva to a pH of from about 9.9 to about 11 irrespective of the starting pH of saliva.

56. A composition of claim 54, wherein said 5-HT agonist is selected from the group consisting of sumatriptan, naratriptan, rizatriptan, eletriptan, almotriptan, zolmitriptan, frovatriptan, and combinations thereof.

57. A composition of claim 54, wherein said carbonate salt is selected from the group consisting of sodium carbonate and potassium carbonate.

58. A composition of claim 54, wherein said bicarbonate salt is selected from the group consisting of sodium bicarbonate and potassium bicarbonate.

59. A composition of claim 54, wherein said metal oxide is selected from the group consisting of magnesium oxide and aluminum oxide.

60. A composition of claim 59, wherein said magnesium oxide is amorphous magnesium oxide.

61. A composition of claim 54, wherein said binary buffer system comprises sodium carbonate and amorphous magnesium oxide.

62. A composition of claim 54, wherein said binary buffer system comprises sodium bicarbonate and amorphous magnesium oxide.

63. A composition of claim 54, wherein said carrier is selected from the group consisting of a binder, a gum base, and combinations thereof.

64. A composition of claim 54, wherein said composition is a dosage form selected from the group consisting of a lozenge, a chewing gum, a chewable tablet, and a dissolving tablet.

65. A composition of claim 56, wherein said dissolving tablet is selected from the group consisting of a slow-dissolving tablet and a quick-dissolving tablet.

66. A composition of claim 54, wherein said oral mucosa is selected from the group consisting of the sublingual mucosa, the buccal mucosa, and a combination thereof.

67. A composition of claim 54, wherein the average particle size of said 5-HT agonist or a pharmaceutically acceptable salt thereof is less than or equal to the average particle size of said carrier.

68. A composition of claim 54, wherein said 5-HT agonist is sumatriptan and said binary buffer system comprises sodium carbonate or sodium bicarbonate and amorphous magnesium oxide.

69. A composition of claim 68, wherein said composition is a lozenge or a dissolving tablet.

70. A composition of claim 69, wherein said composition is administered sublingually.

71. A composition of claim 68, wherein the weight percent of amorphous magnesium oxide is greater than the weight percent of sodium carbonate or sodium bicarbonate.

72. A composition for delivery of a 5-HT agonist across the oral mucosa, said composition comprising:
(a) a 5-HT agonist or a pharmaceutically acceptable salt thereof;
(b) a carrier; and
(c) a binary buffer system comprising a carbonate salt or a bicarbonate salt and a citrate, phosphate, or borate salt,
wherein said binary buffer system raises the pH of saliva to a pH greater than about 9.9 irrespective of the starting pH of saliva.

73. A composition of claim 72, wherein said binary buffer system raises the pH of saliva to a pH of from about 9.9 to about 11 irrespective of the starting pH of saliva.

74. A composition of claim 72, wherein said 5-HT agonist is selected from the group consisting of sumatriptan, naratriptan, rizatriptan, eletriptan, almotriptan, zolmitriptan, frovatriptan, and combinations thereof.

75. A composition of claim 72, wherein said carbonate salt is selected from the group consisting of sodium carbonate and potassium carbonate.

76. A composition of claim 72, wherein said bicarbonate salt is selected from the group consisting of sodium bicarbonate and potassium bicarbonate.

77. A composition of claim 72, wherein said carrier is selected from the group consisting of a binder, a gum base, and combinations thereof.

78. A composition of claim 72, wherein said composition is a dosage form selected from the group consisting of a lozenge, a chewing gum, a chewable tablet, and a dissolving tablet.

79. A composition of claim 78, wherein said dissolving tablet is selected from the group consisting of a slow-dissolving tablet and a quick-dissolving tablet.

80. A composition of claim 72, wherein said oral mucosa is selected from the group consisting of the sublingual mucosa, the buccal mucosa, and a combination thereof.

81. A composition of claim 72, wherein the average particle size of said 5-HT agonist or a pharmaceutically acceptable salt thereof is less than or equal to the average particle size of said carrier.

82. A composition of claim 72, wherein said 5-HT agonist is sumatriptan and said binary buffer system comprises sodium carbonate or sodium bicarbonate and and a citrate, phosphate, or borate salt.

83. A composition of claim 82, wherein said composition is a lozenge or a dissolving tablet.

84. A composition of claim 83, wherein said composition is administered sublingually.

85. A composition for delivery of a 5-HT agonist across the oral mucosa, said composition comprising:
(a) a 5-HT agonist or a pharmaceutically acceptable salt thereof;
(b) a carrier; and
(c) a binary buffer system comprising a metal oxide and a citrate, phosphate, or borate salt,
wherein said binary buffer system raises the pH of saliva to a pH greater than about 9.9 irrespective of the starting pH of saliva.

86. A composition of claim 85, wherein said binary buffer system raises the pH of saliva to a pH of from about 9.9 to about 11 irrespective of the starting pH of saliva.

87. A composition of claim 85, wherein said 5-HT agonist is selected from the group consisting of sumatriptan, naratriptan, rizatriptan, eletriptan, almotriptan, zolmitriptan, frovatriptan, and combinations thereof.

88. A composition of claim 85, wherein said metal oxide is selected from the group consisting of magnesium oxide and aluminum oxide.

89. A composition of claim 88, wherein said magnesium oxide is amorphous magnesium oxide.

90. A composition of claim 85, wherein said carrier is selected from the group consisting of a binder, a gum base, and combinations thereof.

91. A composition of claim 85, wherein said composition is a dosage form selected from the group consisting of a lozenge, a chewing gum, a chewable tablet, and a dissolving tablet.

92. A composition of claim 91, wherein said dissolving tablet is selected from the group consisting of a slow-dissolving tablet and a quick-dissolving tablet.

93. A composition of claim 85, wherein said oral mucosa is selected from the group consisting of the sublingual mucosa, the buccal mucosa, and a combination thereof.

94. A composition of claim 85, wherein the average particle size of said 5-HT agonist or a pharmaceutically acceptable salt thereof is less than or equal to the average particle size of said carrier.

95. A composition of claim 85, wherein said 5-HT agonist is sumatriptan and said binary buffer system comprises amorphous magnesium oxide and a citrate, phosphate, or borate salt.

96. A composition of claim 95, wherein said composition is a lozenge or a dissolving tablet.

97. A composition of claim 96, wherein said composition is administered sublingually.

98. A composition for delivery of a 5-HT agonist across the oral mucosa, said composition comprising:
(a) a 5-HT agonist or a pharmaceutically acceptable salt thereof;
(b) a carrier; and
(c) a binary buffer system comprising a carbonate salt and a bicarbonate salt,
wherein said binary buffer system raises the pH of saliva to a pH greater than about 9.9 irrespective of the starting pH of saliva.

99. A composition of claim 98, wherein said 5-HT agonist is sumatriptan and said binary buffer system is combined with sumatriptan to form a solution just prior to delivery of sumatriptan to the oral mucosa.

100. A composition of claim 98, wherein said 5-HT agonist is sumatriptan and said binary buffer system comprises sodium bicarbonate and sodium carbonate wherein the ratio of sodium bicarbonate to sodium carbonate is from about 2:1 to about 5:1 by weight.

101. A composition of claim 100, said composition delivering a peak plasma concentration within about 1-15 minutes following administration.

102. A method for treating a migraine in a subject in need thereof, said method comprising:
administering to said subject a composition comprising a therapeutically effective amount of sumatriptan or a pharmaceutically acceptable salt thereof, a carrier, and a binary buffer system comprising a carbonate salt and a bicarbonate salt, wherein said binary buffer system raises the pH of saliva to a pH greater than about 9.9 irrespective of the starting pH of saliva.

103. A method in accordance with claim 102, wherein said composition is a solution composition.

104. A method in accordance with claim 103, wherein said binary buffer system comprises sodium bicarbonate and sodium carbonate wherein the ratio of sodium bicarbonate to sodium carbonate is from about 2:1 to about 5:1 by weight, and said composition provides a peak plasma concentration within about 1-15 minutes following administration to said subject.

105. A method for treating a migraine in a subject in need thereof, said method comprising:
administering to said subject a composition comprising a therapeutically effective amount of a 5-HT agonist or a pharmaceutically acceptable salt thereof, a carrier, and a ternary buffer system comprising a carbonate salt, a bicarbonate salt, and a metal oxide, wherein said ternary buffer system raises the pH of saliva to a pH greater than about 9.9 irrespective of the staring pH of saliva.

106. A method of claim 105, wherein said ternary buffer system raises the pH of saliva to a pH of from about 9.9 to about 11 irrespective of the starting pH of saliva.

107. A method of claim 105, wherein said composition delivers'said 5-HT agonist across the oral mucosa.

108. A method of claim 107, wherein said oral mucosa is selected from the group consisting of the sublingual mucosa, the buccal mucosa, and a combination thereof.

109. A method of claim 105, wherein said migraine is selected from the group consisting of a migraine without aura and a migraine with aura.

110. A method of claim 105, wherein said 5-HT agonist is selected from the group consisting of sumatriptan, naratriptan, rizatriptan, eletriptan, almotriptan, zolmitriptan, frovatriptan, and combinations thereof.

111. A method of claim 105, wherein said carbonate salt is selected from the group consisting of sodium carbonate and potassium carbonate.

112. A method of claim 105, wherein said bicarbonate salt is selected from the group consisting of sodium bicarbonate and potassium bicarbonate.

113. A method of claim 105, wherein said metal oxide is selected from the group consisting of magnesium oxide and aluminum oxide.

114. A method of claim 113, wherein said magnesium oxide is amorphous magnesium oxide.

115. A method of claim 105, wherein said ternary buffer system comprises sodium carbonate, sodium bicarbonate, and amorphous magnesium oxide.

116. A method of claim 105, wherein said carrier is selected from the group consisting of a binder, a gum base, and combinations thereof.

117. A method of claim 105, wherein said composition is a dosage form selected from the group consisting of a lozenge, a chewing gum, a chewable tablet, and a dissolving tablet.

118. A method of claim 117, wherein said dissolving tablet is selected from the group consisting of a slow-dissolving tablet and a quick-dissolving tablet.

119. A method of claim 105, wherein said oral mucosa is selected from the group consisting of the sublingual mucosa, the buccal mucosa, and a combination thereof.

120. A method of claim 105, further comprising a 5-HT antagonist.

121. A method of claim 105, further comprising a non-steroidal anti-inflammatory drug (NSAID).

122. A method of claim 105, wherein the average particle size of said 5-HT agonist or a pharmaceutically acceptable salt thereof is less than or equal to the average particle size of said carrier.

123. A method of claim 105, wherein said 5-HT agonist is sumatriptan and said ternary buffer system comprises sodium carbonate, sodium bicarbonate, and amorphous magnesium oxide.

124. A method of claim 123, wherein said composition is a lozenge or a dissolving tablet.

125. A method of claim 124, wherein said composition is administered sublingually.

126. A method of claim 123, wherein the weight percent of amorphous magnesium oxide is greater than the combined weight percent of sodium carbonate and sodium bicarbonate.
